(19) United States
(12) Patent Application Publication
(10) Pub. No.: US 2018/0213046 A1
(43) Pub. Date: Jul. 26, 2018

(54) Organization, Analysis, and Management of Digital Interactions on Networked Computers

(71) Applicant: Shryne Limited, Dublin (IE)

(72) Inventors: Aldongar CHERDABAYEV, Almaty (KZ); Gulbanu Cherdabayeva, Wellesley, MA (US)

(21) Appl. No.: 15/927,292

(22) Filed: Mar. 21, 2018

Related U.S. Application Data
(63) Continuation of application No. PCT/IB16/01439, filed on Sep. 21, 2016.
(60) Provisional application No. 62/221,404, filed on Sep. 21, 2015.

(51) Int. Cl. H04L 29/08; G06Q 50/00; G16H 50/30

(52) U.S. Cl. CPC H04L 67/22; G16H 50/30; G06Q 50/01; H04L 67/306

(57) ABSTRACT

Systems and methods here include computerized ways for copying, importing, synchronizing, analyzing and organizing data, particularly involving communication data from any communication channels. Some embodiments include using a computer to access and import multiple user communication channels over the network, synchronize relationship data from the corresponding channel for each of the designated contact entries with their corresponding identifier(s). Some embodiments may then analyze the synchronized communication data and contact data to provide feedback for users in digestible formats.

### CROSS REFERENCE TO RELATED CASES

This application claims priority under 35 U.S.C. §120 to and is a continuation of PCT international patent application PCT/IB16/01439 filed 21 Sep. 2016, claims priority from U.S. Provisional application 62/221,404 filed 21 Sep. 2015, which are all hereby incorporated by reference in their entirety.

### TECHNICAL FIELD

This application primarily relates to the fields of personal communication data collection, organization, storage, analysis, and management. The application also relates to the field of social networking.

### BACKGROUND

Beginning with the creation of text messaging, personal digital communications have evolved to now span hundreds of platforms for social interaction. The focus of innovation in this domain has so far been on perfecting existing and creating new channels of digital communication. Among their many benefits, the development of digital communications resulted in each user generating a richly recorded personal history within and across these many channels. This scattered history is a biographical document that is comprised of automatically transcribed digital exchanges with everyone a person engages with throughout a lifetime.

The personal use of digital communications across a plethora of channels (including email, text messages, voice calls, voicemail, video calls, instant messaging, social network messaging, other social network interactions, audio and/or visual media shared privately or publicly, in addition to various applications that support activity planning, social event organizing, any form of collaboration, shopping, peer-to-peer payments, and co-sharing of goods and services, etc.) generates data that may be stored within, and used for the purposes of, each channel. This is a technical problem, the organizing and synthesizing of all of these multiple channels of communications that are constantly updated and growing.

Because such data is not aggregated, each person may find it hard to capitalize on his or her digital history. Moreover, lacking the technical solutions to merge these data into a single repository makes it more challenging to archive and preserve these data and the memories they contain. Multiple threads of communication also make it a challenge to search for and view past interactions. The disconnected threads may be stored in incompatible formats, which could make any user-made solution prohibitively difficult.

The novelty and complexity of the issues requires a new cultural and technological paradigm. The following invention proposes technological solutions that organize personal communication data to solve the existing issue and reimagine the future value of such data.

### SUMMARY

Systems and methods here include computerized ways for organizing data, including, using a computer processor in communication with a data storage server and a network, the computer processor configured to, communicate with multiple channels over the network, wherein each of the multiple channels includes stored relationship data and contact information for each of multiple contact identifiers, receive a designation of each of multiple contact identifiers, receive an indication of a corresponding channel, from the multiple channels, for each of the designated contact entries, synchronize relationship data from the corresponding channel for each of the designated contact entries with their corresponding identifier at the channel, wherein the synchronize includes, communicate with the corresponding channel through a respective channel interface, copy, import, synchronize and retrieve the stored relationship data and the contact information for each of the multiple identifiers, store the retrieved relationship data and contact information for each of the multiple identifiers, relate the stored relationship data and contact information to the respective designated contact identifier, and update the stored relationship data by retrieving new relationship data and contact information from the respective channel on a regular or irregular basis. In some embodiments, regular synchronization is periodic scanning for new data. In some embodiments, irregular synchronization is triggered by an event. In some embodiments, an event is a user-initiated syncing process or new data in the channel. In some embodiments, a channel is a digital communication channel. In some embodiments, a channel is a third party social networking web site. In some embodiments, the match is by user selection or automatic by the system. In some embodiments, the relationship data is at least one of a content name and a contact detail.

Alternatively or additionally, some embodiments include aspects of an Archive Feed. In some embodiments, the systems and methods may further cause display of an archive feed related to each of the multiple identifiers. In some embodiments, the archive feed includes a record of all of the communications with each of the multiple identifiers displayed chronologically. In some embodiments, the display of the archive feed maybe edited by a user including a favorite indication, a delete of communication data, and adding a caption. In some embodiments, the system is further configured to allow a search of the communications data and the multiple identifiers. In some embodiments, the system is further configured to allow the display to be filtered thereby causing display of only filtered communication data. In some embodiments, the filter is at least one of a time filter and a channel filter. In some embodiments, the systems and methods may further allow a user to freeze selected communication data with a timer for a time, thereby stopping the system from being able to cause display of the frozen communication data for the timer time. In some embodiments, the systems and methods may password protect the frozen communication data. In some embodiments, the selected communication data is based on at least one of an identifier, a time, and a channel. In some embodiments, the data storage server includes the channel data storage. In some embodiments, the channels are at least one of, an email, a text message, and an instant message. In some embodiments, the computer is further configured to, store a duplicate copy of the relationship data and contact information from the corresponding channel in the data storage server.

Alternatively or additionally, some embodiments include contact and type setup as well as data synchronizing. Systems and methods here may include embodiments for organizing data from communications, the system including using a computer processor in communication with a data storage server and a network, the computer processor configured to, create and store in the data storage server, a user profile for a user, receive a list of contact identifiers and related relationship data regarding each contact identifier in the list and the user, store the received list of contact identifiers and related relationship data in the data storage server, connect to multiple channels over the network, identify contacts within each of the multiple channels, wherein the identified contacts are included in the received list of contact identifiers, copy, import, or synchronize contact information for each identified contact from the multiple channels, synchronize the copied, imported, or synchronized contact information from the multiple channels with the received list of contact identifiers, and save the synchronized information and associated contact identifier on the data storage server. In some embodiments, at least one of the multiple channels is a third party website, and the connection to the channel includes access via a user login corresponding to the user. In some embodiments, the data storage server is the channel data storage server. In some embodiments, the channel is at least one of email, text message and instant message. In some embodiments, the channel is a social media website post. In some embodiments, the contact identifiers are usernames. In some embodiments, the computer is further configured to generate a user interface including contact information.

Alternatively or additionally, some embodiments include using filters and grouping conversation data for display. Systems and methods here may include embodiments for organizing data from communications, the system including, using a computer processor in communication with a data storage server and a network, the computer processor configured to, receive data over the network related to communications from multiple channels over the network, filter the communication data to extract details about the communications, group the communication data from the conversations according to the extracted details, and cause display of a subset of the extracted, grouped details in a user interface. In some embodiments, the details from the communication include at least one of, file/data type information, data originator, recipient, date and time, text content, and media content. In some embodiments, the text content is at least one of a message body, an automatic signatures, a previous message, and other automated element. In some embodiments, the communication data is grouped regardless of channel. In some embodiments, the display of the extracted data includes sender information. In some embodiments, the multiple channels are third party websites. In some embodiments, the extracted details include a channel or time period. In some embodiments, the extracted details include key words in the communication data. In some embodiments, the communications are at least one of text messages, instant messages, or email. In some embodiments, the computer is further configured to generate a graph and send data regarding the generated graph for display on a user interface. In some embodiments, the graph includes data from the communications data. In some embodiments, the graph includes data from the extracted details.

Alternatively or additionally, some embodiments include using data analytics with the communication data. Systems and methods here may include embodiments for archiving digital information, including using a computer including a processor, memory, and data storage server in communication with a network, the computer configured to, store data regarding contacts and communications for a user in the data storage server, store an indication of a relationship between the user and each stored contact in the data storage server, synchronize data over the network by importing, synchronizing or copying user communication data from a channel using a user channel account, create a user profile for the user and the stored indications of relationships, analyze the user communication data to determine a macro communication pattern, analyze the user communication data between the user and each stored contact to determine respective micro communication patterns. In some embodiments, the communication patterns include at least one of, number of words per interaction or message, amount of time it takes for the user or contact to reply to messages, the rate of response to messages, and average time of day the interactions happened. In some embodiments, the system is further configured to cause display of graphs of the communication data including at least one of, number of conversations, messages, media files in the archive, and storage size. In some embodiments, the communication patterns include linguistic analysis, including analyzing words in the communications with lists to determine at least one of, a tone of communication, words per interaction or message, keywords used, ideograms and emoticons used. In some embodiments, the communication matters include message analysis, including counting messages volume, percentage of conversations initiated by a sender, number of words per message and number of messages per conversation, and average response times. In some embodiments, the channel is at least one of a text message, an email, and an instant message. In some embodiments, the computer is further configured to analyze he communication data to determine a causality of an event. In some embodiments, the computer is further configured to determine a forecast based on the causality of the event. In some embodiments, the computer is further configured to create a user matching profile for the user based on aggregated analytical outputs from the user account and communication patterns. In some embodiments, the computer is further configured to, compare the user matching profile with other user matching profiles to determine a match, send information about the matching profiles to the respective users over the network. In some embodiments, the information sent about the matching profiles includes contact information for the respectively matched users. In some embodiments, the computer is further configured to, compare the communication data for the user to a set of metrics to determine at least one social heath indicator. In some embodiments, the social health indicator includes at least one of, tone, conflict, trust, stability, enjoyment, depression, anxiety and honesty. In some embodiments, the computer is further configured to allow a user to share the social health indicator over the network. In some embodiments, the data storage server includes data stored from the channel.

### BRIEF DESCRIPTION OF THE DRAWINGS

FIG. 1 is a diagram that describes the archive creation process and the primary components of the unified storage solution according to certain embodiments herein.

FIG. 2 is a diagram that describes the data import process for multiple channels according to certain embodiments herein.

FIG. 3 is a diagram that describes the hardware implementation according to certain embodiments herein.

FIG. 4 is a diagram that describes the System architecture according to certain embodiments herein.

FIG. 5 is a diagram that outlines the System's primary data processing operations according to certain embodiments herein.

FIG. 6 is a diagram that describes the filtering process for an example text record according to certain embodiments herein.

FIG. 7 is a diagram that describes the filtering process for other types of direct communication data according to certain embodiments herein.

FIG. 8 is a diagram that shows a general embodiment of a “conversation” unit and compares clustering methods according to certain embodiments herein.

FIG. 9 is a diagram that displays a general strategy for displaying messages in a group conversation according to certain embodiments herein.

FIG. 10 is a flowchart that describes the conversation creation algorithm, which matches each record to the correct conversation according to certain embodiments herein.

FIG. 11 is a diagram that demonstrates how to best organize conversations in a single timeline according to certain embodiments herein.

FIG. 12 is a flowchart that describes the preparation of analytics data according to certain embodiments herein.

FIG. 13 is a diagram that describes how the user creates and syncs an archive according to certain embodiments herein.

FIG. 14 is a diagram of the archive feed view according to certain embodiments herein.

FIG. 15 is a diagram of the archive filter view according to certain embodiments herein.

FIG. 16 is a flowchart that describes the freeze archive feature according to certain embodiments herein.

FIG. 17 is a diagram of the library view according to certain embodiments herein.

FIG. 18 is a flowchart that describes the channel management process according to certain embodiments herein.

FIG. 19 is a diagram that describes the general setup of a shared archive according to certain embodiments herein.

FIG. 20 is a diagram that describes the analytics sequence through all components according to certain embodiments herein.

FIG. 21 is a diagram that describes the general elements of an archive analysis according to certain embodiments herein.

FIG. 22 is a diagram of the general user profile interface according to certain embodiments herein.

FIG. 23 is a diagram of the library analysis interface according to certain embodiments herein.

FIG. 24 is a diagram that describes the general setup of system analytics according to certain embodiments herein.

FIG. 25 is a diagram of the relationships manager interface according to certain embodiments herein.

FIG. 26 is a diagram of the general user matching process according to certain embodiments herein.

FIG. 27 is a diagram of a primary social health indicator according to certain embodiments herein.

FIG. 28 is a diagram of social health tracking according to certain embodiments herein.

FIG. 29 is a diagram of social health tracking according to certain embodiments herein.

FIG. 30 is a diagram of a social health analytical model according to certain embodiments herein.

FIG. 31 is a diagram that compares the decentralized and centralized embodiments of social data storage server and sharing according to certain embodiments herein.

### DETAILED DESCRIPTION

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the systems and methods described here, to these particular embodiments. In other instances, well-known data structures, timing protocols, software operations, procedures, and components have not been described in detail so as not to unnecessarily obscure aspects of the embodiments of the systems and methods described here.

### Overview

These disclosure here includes details of various embodiments, which alone or in combination with one another may create a paradigm for the utilization of computerized personal communication records and networked interactions. Such systems and methods may be used to improve the way systems preserve data and how users interact with such data. Communication data may be analyzed and processed in order to display various charts, graphs and data output for users. Data may be copied, imported, or synchronized from user profiles in various online settings such as social networking sites, and that data may be analyzed and processed in order to make connections with other users. It should be noted that the systems and methods here may be used to aggregate many multiple channels of communication from one user, even over multiple accounts or channels, and combine the analysis of the many communication channels for that user. Thus, the analysis for a user may include analysis for multiple channels of communication by that user, over multiple channels and accounts. This may benefit the user and the system, by drawing together channels that had never been analyzed in the aggregate and allow both user interaction with the data, as well as system analysis of the data.

The systems and methods described here include systems (“System” 0 and methods, can be understood as a cluster of interdependent technologies which alone or in any combination among one another may be used advantageously as described herein. The System shall be described in six parts: 1. Unified Archiving, 2. Data Processing, 3. User Interface, 4. Analytics, 5. Relationships Manager, 6. Centralized Social Data Storage Infrastructure. Each technology benefits from the implementation of the preceding steps—for example, analytics may benefit from implementation alongside unified archives, as the feature takes advantage of a more complete data set. The systems and methods described here may thus include both the methods of use and systems that utilize these technologies and each individual technology that is created for the system. Moreover, each individual part represents an inventive step that may function as a standalone technology in a different context, or as combined with any of the other individual parts described herein.

It should be noted that user devices which could be used to interact with the System, send and receive data to the System, and otherwise process data, store data and analyze data could include any of various computers such as but not limited to smartphones, laptops, tablets, phablets, wearable computers such as glasses and watches, or any other kind of computing device. Such user devices may communicate via various wired and unwired communications methods with the Systems here including but not limited to cellular, WiFi, Bluetooth Low Energy, Near Field Communications, pico cell, nano cell, or any other communication type. Such communication could be to any kind of network such as the Internet through which any of various back end servers, storage devices and services could be accessed, as well as other user devices.

The Systems described here could include any number of computer components such as server hardware including processor(s), memory, data storage, network interface(s), and any number of software programs stored in the memory and executed on the processor(s) to send, receive and otherwise process data.

Part 1: Unified Archiving

The first part of the systems and methods described here involves the creation of a unified repository of communication and communication-related data for one relationship or social group. As the relative share of digital social interactions is increasing, so is the importance of specialized archiving methodologies. Given that social data is inherently relational, the System organizes each user's entire social history as a series of individual relationship archives.

Archives may include an organized data collection that contains all records from a single relationship (or a group of relationships).

Data may include records of direct digital communications, online social activity, or any other records that chronicle social interactions online and offline. Includes, but not limited to, direct records (messages, calls, media sharing, social network interactions, etc.), secondary records (records of shared activities, interests, analytics, etc.), and manual records (diaries, notes, calendars, contact lists, etc.).

The user begins the archiving process by designating the contact(s) for whom the archive is being created. Defining the nature of a relationship (or group of relationships) remains the prerogative of the user, however, the user interface may provide relationship categories and subsequent customization (as described in Parts 2 and 3).

Archives may be created by synchronizing all relationship data from multiple channels into the System database. In addition to preexisting data (“relationship history”), archives sync and store ongoing, live communications and other data that are imported periodically, either using automatic syncing or by having the user either manually upload the data or launch the syncing command for the channel(s).

Databases may include a collection of information that is organized to facilitate information access and management. In this System, the database stores all user data, which include user communication records, profiles, and preferences. The database may be set up as a cloud server to make the data more accessible, a local server to make the data more secure, or a combination of both. This description will focus on the cloud server setup for example only.

Sync may include creating a duplicate copy of the data from a channel that is stored in the database. The syncing setup depends on the channel, so data syncing may be regular (periodic scanning for new data) or irregular (triggered by events: user-initiated syncing process, the appearance of new data in the System). Furthermore, the System may allow for the automatic or user-initiated removal of source data from the channel upon successfully syncing copies to the database.

Channels may include digital communication or other communication or social activity record creation, such as a social networking third party website, including any form of telecommunications, messaging, social websites and applications, other websites and applications that record interactions, various file types that record interactions (online and offline), and other related technologies.

One example benefit of having the data duplicated into a separate database is the reduction of archival risks. The System presumes the paramount importance of preserving personal records in their original form (“original data”), and thus one of the objectives of the systems and methods described here, is to protect and fully archive such records. Having only one copy of the data in the original channel, even with backups of the data to other channels, may expose the user to certain risk factors, including:

The third party platform that hosts, maintains, or otherwise controls the data on the user's behalf may accidentally or intentionally delete, downsample, edit, modify, convert, corrupt, or otherwise erode the archival use of the data.

The user may accidentally or intentionally delete, downsample, edit, modify, convert, corrupt, or otherwise erode the archival use of the data.

The technology in use may become unsupported or unavailable, making the data stored in that channel inaccessible in the future.

The data format in use may become unsupported, modified, corrupted or otherwise changed, eroding the archival use of the data.

The third party that created and maintained a channel may cease to exist and/or discontinue its support of the channel's service in full or in part, endangering the data for archival use.

The terms under which the channel operates may change, creating barriers to obtaining the data under original or otherwise acceptable terms, increasing costs and risks to reclaiming the data.

The storage solution for the original data may fail in any number of ways, causing the loss of the only copy of that data and eliminating the possibility of its archival use.

The above risks can be mitigated by a storage solution that is designed with the sole purpose of preserving and archiving personal communication data. The following subsections explain the archive creation and data syncing processes of the System.

1.1 Archive Creation

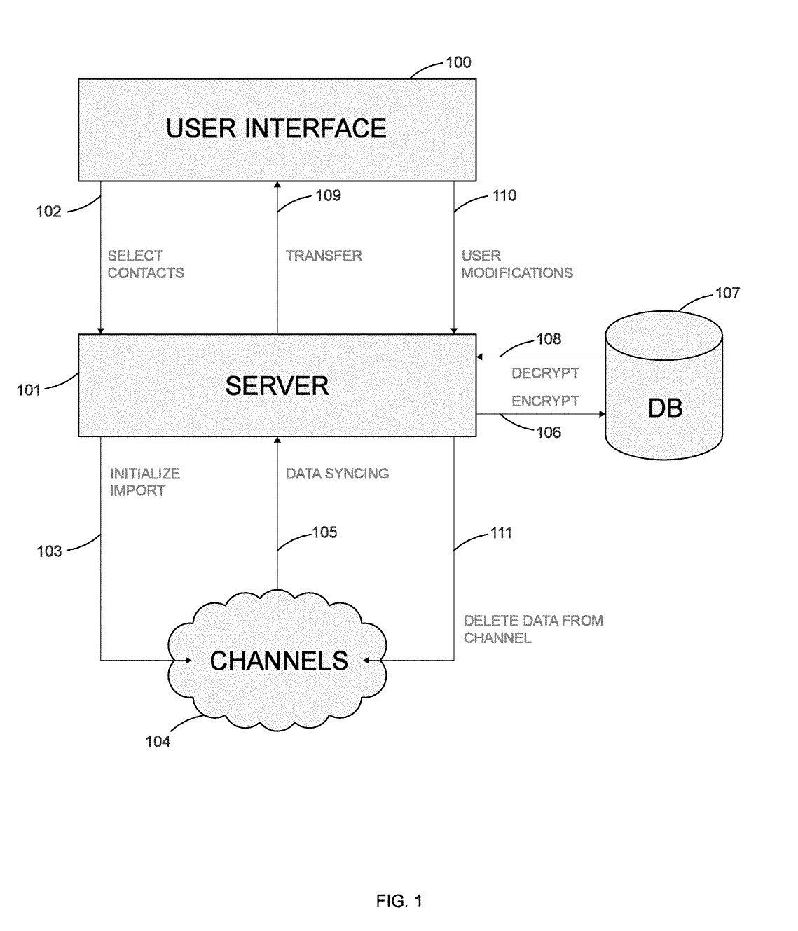

FIG. 1 is a system diagram that describes the example archive creation process and the primary components of the unified storage solution. To create an archive, the user launches the user interface 100 (a mobile, web, or desktop application), which is connected to the System server 101. In the user interface 100, the user selects one or more contacts 102 for the archive. The user selects the channels 104 to sync data from and the server 101 initializes the import 103. The data that relates to the user's relationship with the archive contact(s) is then synced 105 to the server 101 to be encrypted and stored 106 in the database 107. The data syncing 105 can happen on a one-time and/or ongoing basis, depending on the channel.

The data is further processed by the server 101 (described in Part 2). For the purpose of displaying the archive data to the user, the data is optimized for the user interface, decrypted 108, and transferred securely 109 to the user interface 100. The user accesses the data in the user interface 100 and can further modify 110 the data or apply additional settings via the user interface 100. These modifications 110 include deleting data, modifying display settings, adding a record to a “favorites” list, applying filters, etc. (described in Part 3), and the modifications are recorded in the server 101 for future display requests.

Upon creating a secure copy in the database 107, the System gives the user an option to delete 111 the original file(s) from the channels 104 (where technically possible and permitted). The user may set their settings to automatically delete such data after successful syncing. Alternatively, data deleted by the user in the user interface 100 may also retained in its original form in the database 107 as an archival backup, but not transferred to the user interface 100 as per the user's preferences.

1.2 Multi-Channel Syncing

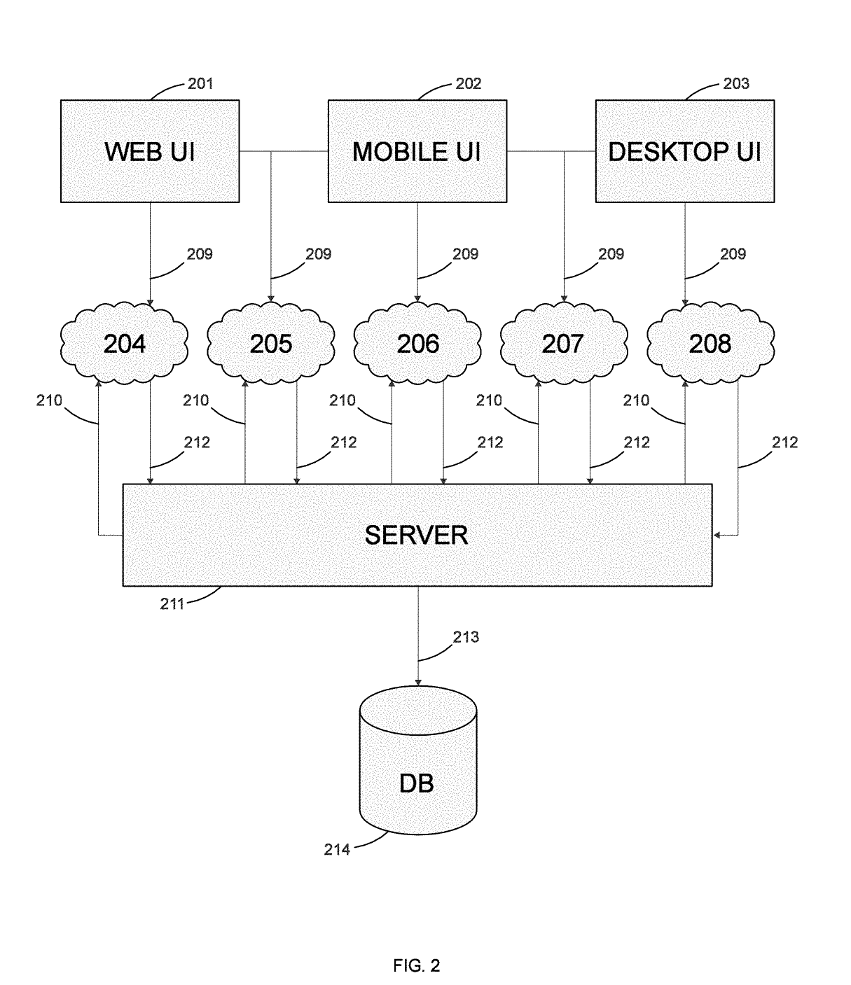

FIG. 2 is a diagram that describes the data import process for multiple channels. The System is designed for maximum channel support. In practice, however, it is unlikely that all user interfaces (mobile 201, web 202, and desktop 203 applications) will be equally able to access and sync data from every channel. Given the restrictions, the user may require one or more user interface 201-203 to sync channels 204-208.

The syncing process begins with the user (manual syncing) or the server (automated/periodical syncing) sending a request 209 to the channel. The request 209 initiates a connection to the channel's application program interface (API), or implies some form of access to the channel by launching it (or accessing its information via the user interface 201-203) and initializing a download process, the specifics of which may vary by channel. The request 209 may attempt to match the information related to the relationship(s) in the archive (the archive contact's name and contact information), which is either matched automatically by the server, manually by the user, or a combination of the two methods (described in more detail in 3.1). Each channel therefore poses a unique challenge, but the System is well-positioned to maximize such integrations using multiple interfaces 201-203.

For example, the channel type 204 can only be accessed via the web application 201 and is not available for syncing using mobile or desktop applications. The more frequent scenarios are channel types 205 and 207, which may be supported by an API that can be accessed via both mobile and web (and usually desktop) applications, as the connection may be made server to server, which may not be limited by a user interface. On the other hand, channel types 206 and 208 are restricted to the mobile and desktop user interfaces, respectively. These channels could include local media (such as photos) that are stored locally, and are therefore only accessible via the application supported by the storage device. In this case, the user (possibly manually) identifies and uploads the data.

Another possible scenario is a channel with no API and with no reliable user access to the data (such as text message backup files or any form of packaged archive). In most cases, this is a channel type 208, which relies on the features and processing power of a desktop or laptop computer to store or access the local data. When syncing channel type 208 the user would rely on the desktop application 203 to parse and sync the data (user input may be required to identify the correct data, depending on the accessibility of archive contact information or details in the data archive). This parsing and syncing feature could be unique to the desktop application, but can also be added to the web application, assuming data processing and potentially large data transfers are practical.

The aforementioned scenarios and embodiments are only a sample of all possible combinations of channels and user interface strategies. The underlying principle of the System is to equip each user with the appropriate interface to maximize their access to personal data that is stored in external channels. Therefore, any and all methods of integrating the System with any channels share the core objective of maximum user access, despite practical variations of their embodiments.

Finally, once the user or the server identifies the data in the channel that matches the intended relationship archive, the data is transferred 210 from the channels 204-208 to the server 211. The server 211 encrypts and stores 212 the data in the database 213, completing the syncing process.

1.3 Hardware Implementation

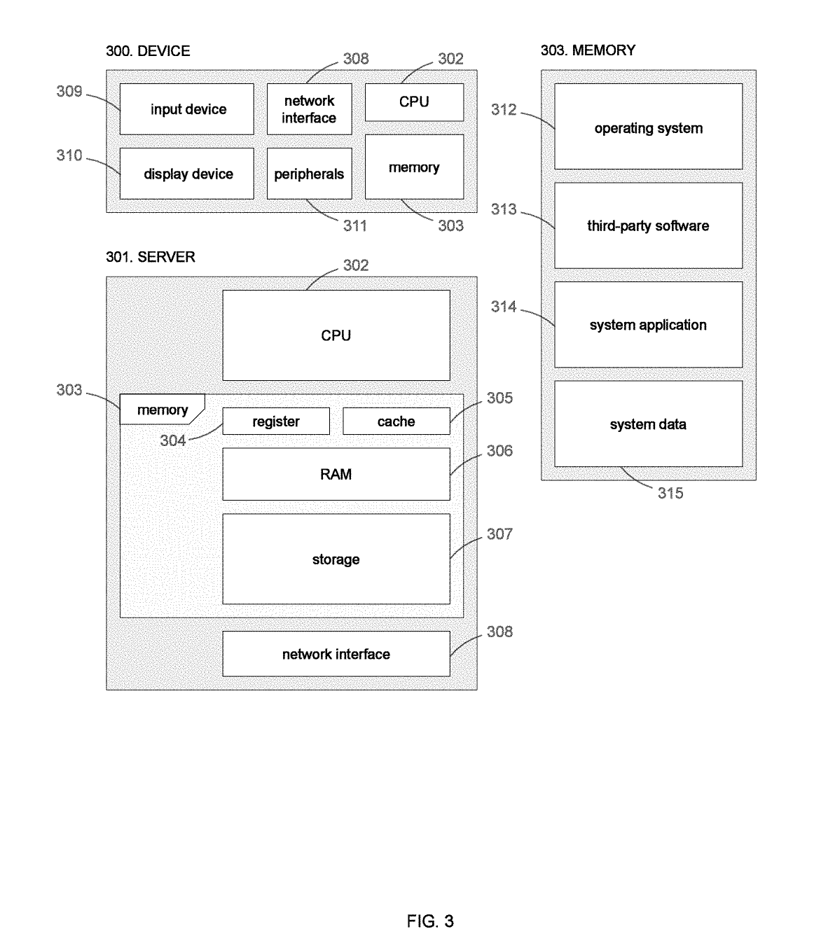

FIG. 3 is an example hardware diagram used to implement certain example embodiments disclosed herein. The System may include a device 300 (any suitable user computing device, such a computer or smartphone) and server 301. Both the device 300 and server 301 could be built from similar components, however server(s) 301 may offer greater performance as they utilize more efficient (and/or multiple) components. Both device 300 and server 301 may consist of the following components:

Central processing unit (CPU) 302 to perform software operations. The relative number of operations may depend on the amount and efficiency of CPU components 302. Server 301 could include multiple CPU 302 units that may be significantly faster than device 300 CPU units to simultaneously handle a large number of users.

Memory 303 to store temporary and/or long term data, which may consist of registers 304 and caches 305 (the most efficient memory type), random access memory (RAM) 306 (for improving data reading and writing speed and storing temporary data), and storage 307 (for temporary, heavy, or long-term data).

The network interface 308 (to allow communication with a different device for the same type of interface, such as WiFi, bluetooth, radio, etc.).

User device 300 may also include the input device 309 (embedded or included as peripheral to allow the user to interact with the System), display device 310 (embedded or included as peripheral to present the System interface prepared for the user device 300), and any other peripherals 311.

FIG. 3 also describes how data is organized inside memory 303. Memory 303 is physically distributed over separate parts. However, the operating system 312 allows memory 303 to be perceived and managed as a single unit. Memory 303 also comprises third-party software 323 and the System software (System application) 314 described here. The operating system 312 loads the System application 314 into memory 404 and the System application 314 loads and operates on the System data 315.

1.4 System Architecture

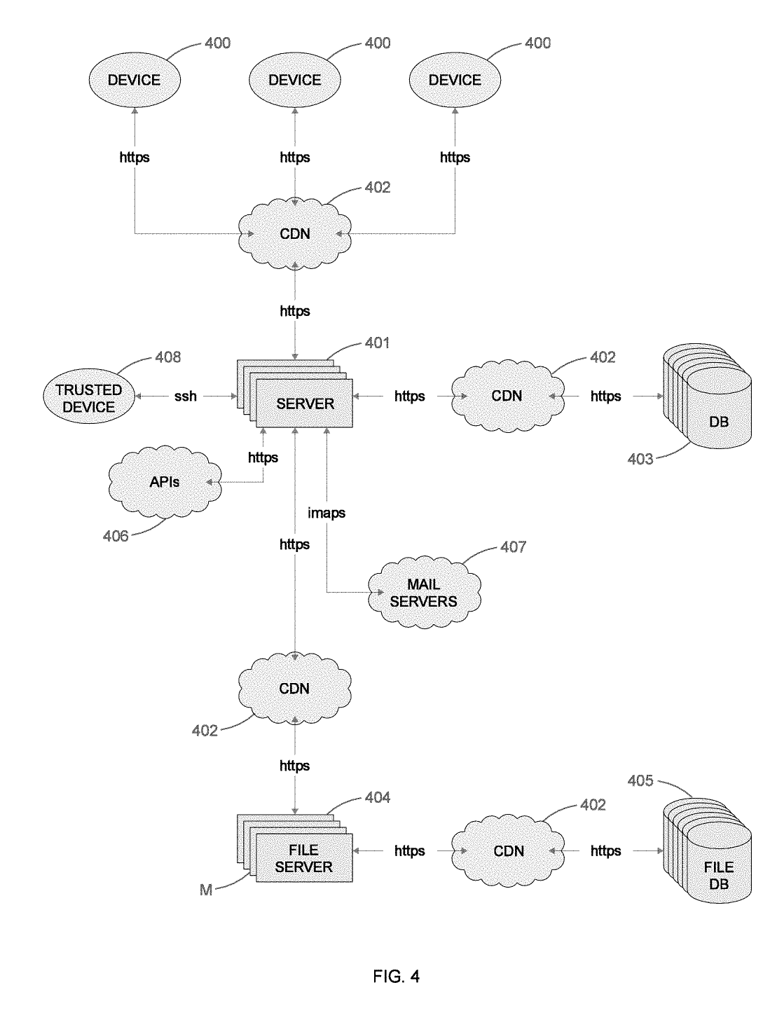

FIG. 4 is a diagram that describes an example System architecture used to implement certain example embodiments disclosed herein. The user device 400 (which runs the mobile, web, desktop, or other user application) sends requests to the server 401 API over the internet network using Hypertext Transfer Protocol Secure (HTTPS). The request is received by a content delivery network (CDN) 402, which selects the optimal server for the device location and/or balances traffic. The System may utilize a CDN service 402 for some or all network connections.

The server 401 can communicate with databases 403 (which store data used by the System), file servers 404 (which hold their own file databases 405 prepared for efficient managing large files, that can be used in our system), third-party APIs 406 (for collecting and/or processing the data), mail servers 407 (for collecting and/or processing the email data), and other components.

The System may allow a direct connection to the server 401 over a Secure Shell Protocol (SSH). However, for security reasons this method may only be allowed for trusted devices 408. This description presents only preferred protocols, but the System may also utilize less secure protocols (e.g. HTTP instead of HTTPS) or different protocols (e.g. SMTPS instead of IMAPS).

Part 2: Data Processing

Syncing data from incompatible channels requires a series of processing operations to make the data usable in the System. These operations identify and correctly organize each record to facilitate the subsequent functionality of the System, including data display and analysis.

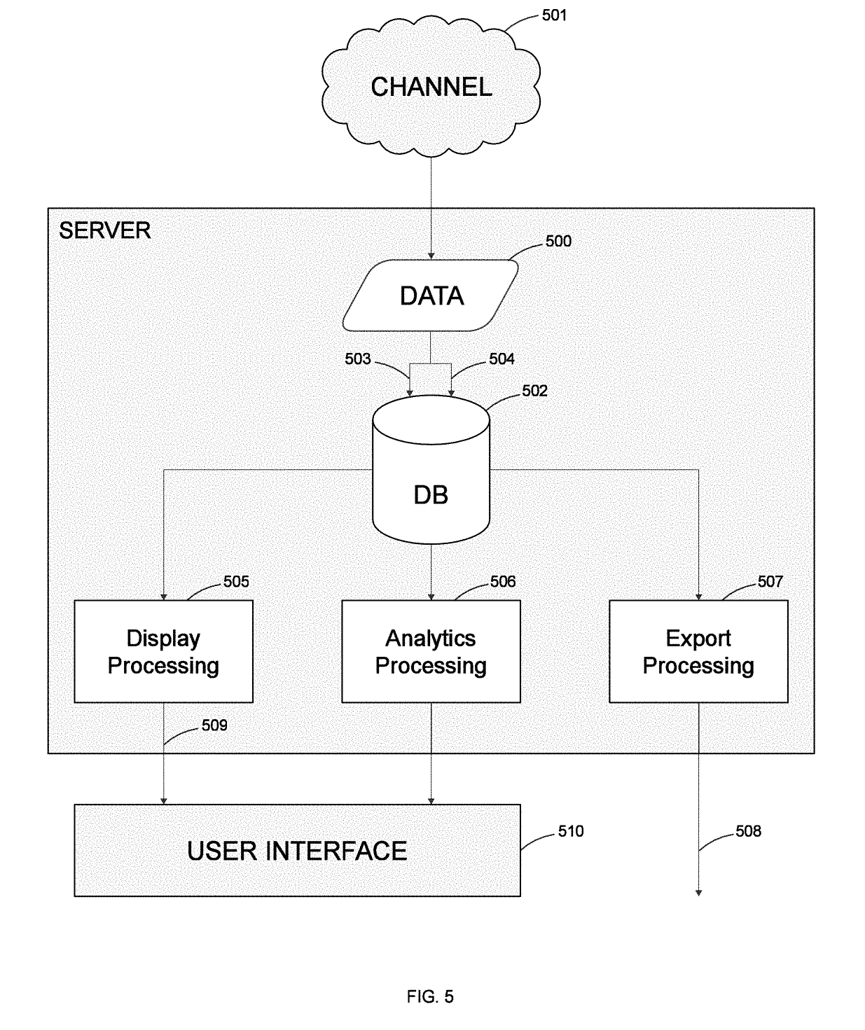

FIG. 5 is a diagram that outlines the System's primary data processing operations. The original data 500 that has been imported in its original format from external channels 501 into the database 502 may require additional processing for further utilization. The System preserves an untouched copy of the original data 503 in the database 502, therefore all data operations may either pull out a working copy of the data 504 or utilize non-destructive operations on the original data 503. The working data is further processed for display 505, analysis 506, and export 507.

The System helps the user regain control over their personal communications data, and therefore a copy of the original data 500 may be downloaded 508 by the user anytime for local storage or other purpose. The data may thus be packaged for exporting 507 either as a single file (for example, a Portable Document Format (.PDF)), a series of files, a folder that organizes and maintains original files separately, and/or any other user-friendly format.

2.1 Processing Data for the User Interface

In order to process the raw data for display purposes, the System shall incorporate a number of operations to optimize and improve the data's readability for the user. Using either a separate copy 503 or non-destructive operations 504 the data undergoes a cleanup and conversion process 505 to filter out superfluous information and/or to format it for the user interface 510.

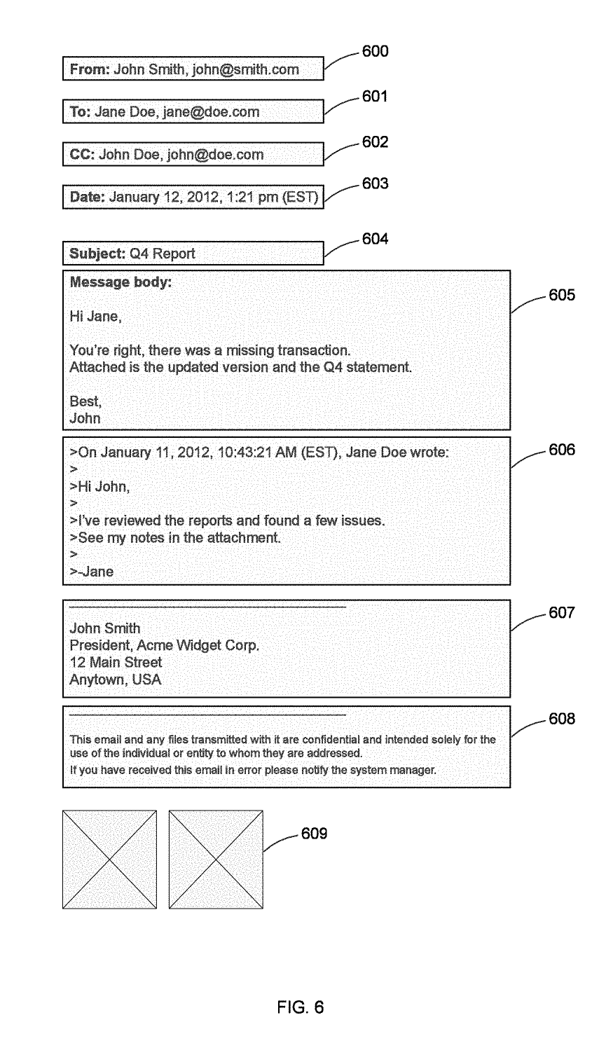

FIG. 6 is a diagram that describes the filtering process for an example text record. The diagram shows standard elements included in an email message, however a subset of these elements can be present in other direct correspondence data (such as text messaging and social network posts). Metadata such as sender 600, recipient 601, date and time 603, message body 605 and/or file attachments 609 may be included in all direct correspondence channels, and the following filtering process can be modified for these channels as well.

When processing direct correspondence data, the System extracts the message body 605 from every message, which may be the most important record. The filter may remove data such as automatic signatures 607, previous messages 606, and any other automated elements 608.

Additional information is extracted to assist in the organization and better display of the messages in the archive. The message sender 600 and recipient 601 information is extracted and tagged as either “archive contact(s)”, “user”, or “third-party participant” (in which case original sender information is retained—see 2.3). The message sender is therefore correctly attributed in the archive. The date and time information 603 is used to place the message in the correct position in the archive. For emails, the message subjects 604 are retained and used to label email conversations in the archive. Any file and media attachments 609 are also extracted and made accessible to the user, either visually in the archive (photos or other media), or made available for download.

Once all elements of the original message have been extracted and tagged, the System can optimize the display of the message to the user by reducing the amount of noise (repeating or unnecessary metadata, irrelevant information, etc.), and also by improving the formatting in the user interface (see 3.2).

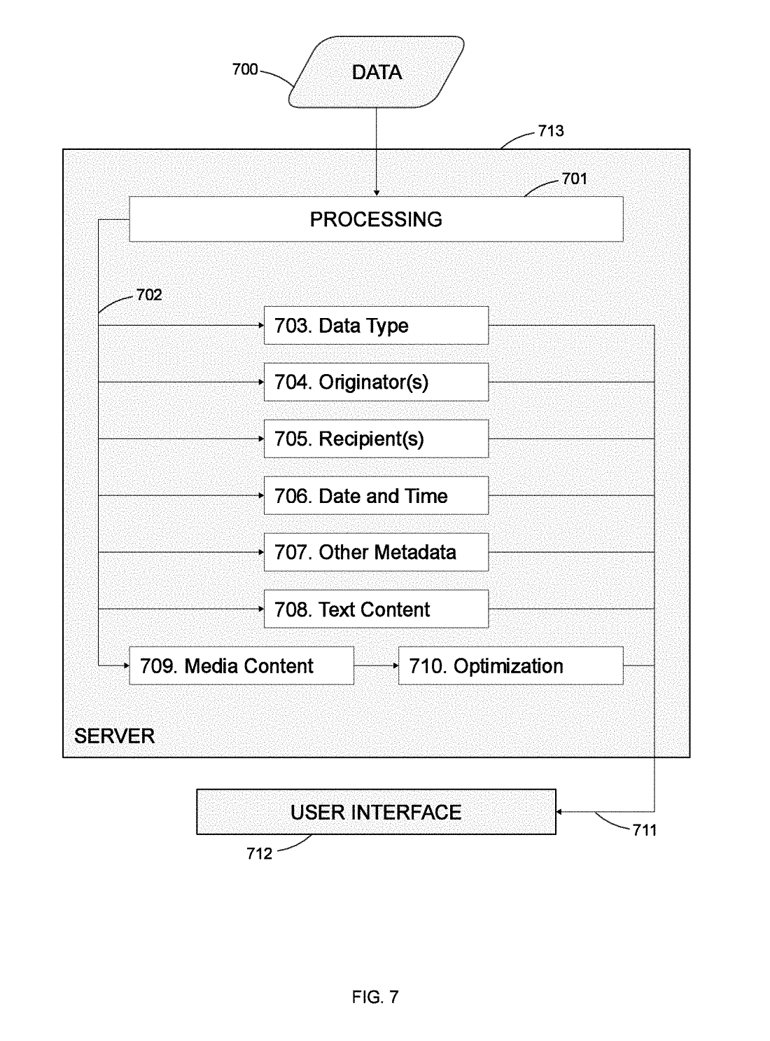

FIG. 7 is a diagram that describes the filtering process for other types of direct communication data. These types of data 700 include records of interactions that are not transcribed, including call logs, voicemail, shared media files, social network activity, and other online activity. These data 700 usually need to be filtered and formatted (processed) 701 for standardized display in the user interface 712. The goal of the processing 701 is to retain only the essential content that contributes to the relationship archive. The System thus extracts 702 file/data type information 703, data originator (the sender, caller, creator, etc. of the record) 704, recipient 705, date and time 706, other metadata 707, text content 708, and media content 709. Post-processed data retains tagged records (following the System standard), is formatted for standardized display, and transferred 711 to the user interface 712. In the case of media content (such as photos and videos) 709 the data is converted, downsampled, or otherwise optimized 710 for high-quality display and quick delivery to each particular user interface 712.

2.2 Conversation Clustering for the Archive Feed

To maximize clarity, the System may organize the synced data chronologically in each user interface. The data may be further subdivided into “conversations”, data that is clustered by channel and/or time period. For example, all text messages from the same time period (e.g. a day) could be grouped into one conversation unit. A more robust strategy defines time periods by analyzing the contents of messages for markers that signal the start and end of each discrete interaction. The goal of the conversation unit is to identify natural breaks in each communication event to better demarcate them.

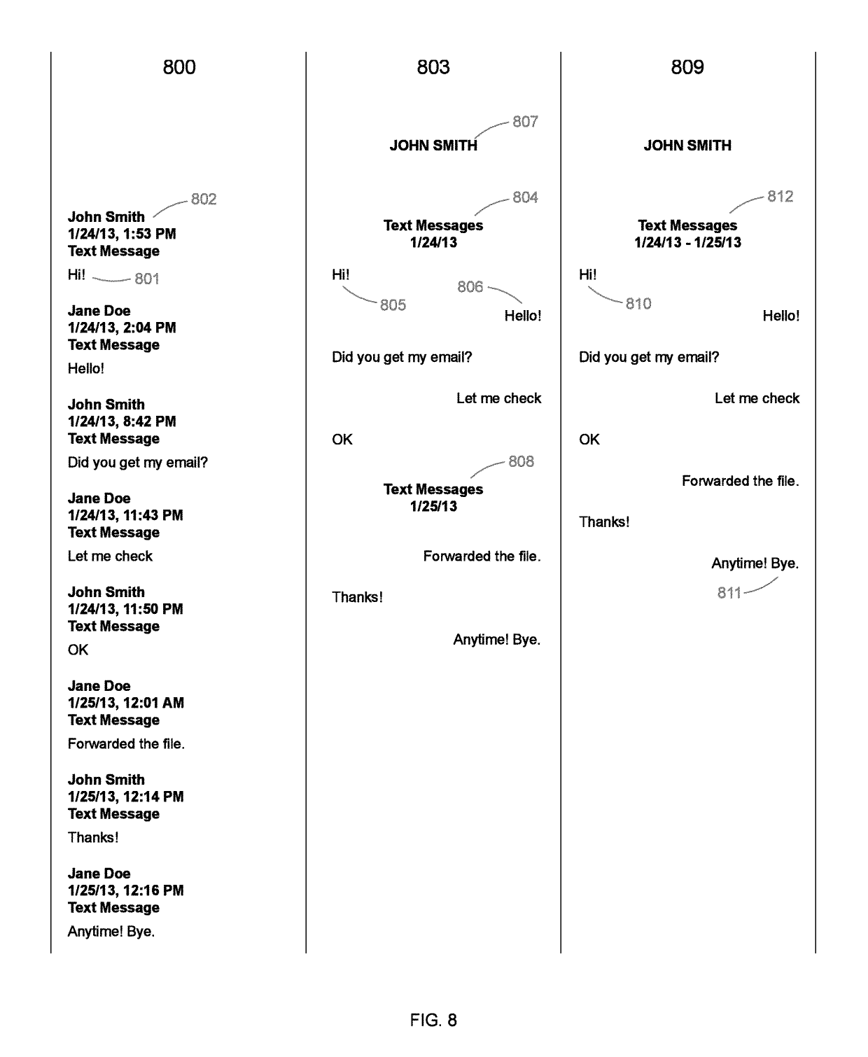

FIG. 8 is a diagram that shows a general embodiment of a “conversation” unit and compares clustering methods. This diagram is using an example text message format, but the logic of conversation clustering applies to every other data type. This diagram displays a single complete interaction between the user Jane Doe and the only archive contact, John Smith.

An example method of assigning metadata to each record creates significant redundancy. In the pre-clustered view 800, each message 801 is preceded by its metadata 802, which includes sender name, date and time, and the channel of origin.

Thus, repeating metadata can be avoided if the messages are clustered into conversation units, as shown in view 803. This clustering method uses a fixed time period, for example only conversations from the same channel on the same day may form a conversation unit. As a result, metadata needs to be included only once per conversation, for example, the date and channel 804 at the beginning. The time information may be included for each message, but could be redundant, as the conversation is organized chronologically.

Sender names may require a more complex strategy. Giving each sender type a unique graphic location removes the need for sender names in archives with one contact. For example, the separation of messages by sender (archive contact on the left 805, user on the right 806) follows an example text message interface and requires no name labels (with two participants). Applying a separate color for messages from the archive contact is another option to make the message sender more obvious. Thus, in case of a single archive contact (one relationship in the archive), showing the contact's name 807 at the start of the archive would be sufficient.

However, in cases of multiple archive contacts and group conversations, the sender names may need to be included with each record. Maintaining a separate placement of user's message-based records on the right side eliminates the need to add the user's name to those records. However, the records from the archive contacts and third party participants may need to be labeled (see 2.3).

The limitation of the fixed-period clustering 803 is the potential inaccuracy in identifying conversation endpoints. FIG. 8 shows a single complete interaction, but in view 803 the conversation is split into two units 805 and 808, because the messages occurred on multiple days.

The third view 809 shows a “smart” clustering method that does not follow a fixed time period, but analyzes the content of the messages and/or the patterns of interactions. For example, the System may identify certain keywords (like “hi” 810 and “bye” 811) as endpoints. In addition to linguistic algorithms, the System may include an analysis of the user's communication patterns, as well as example interactions within this relationship to infer when the conversation ends (with or without linguistic markers), for example, when there's a delay between messages. In this case, the conversation spans two dates, which can be reflected in the conversation metadata 812. This clustering method 809 thus avoids the multi-day spillover effect and is more effective at identifying correct conversation endpoints.

Other clustering methods are possible, including those that display any type of metadata with any frequency (each record, each conversation, etc.). Furthermore, the System can let the user hide or show these metadata according to their preferences.

2.3 Group Conversations

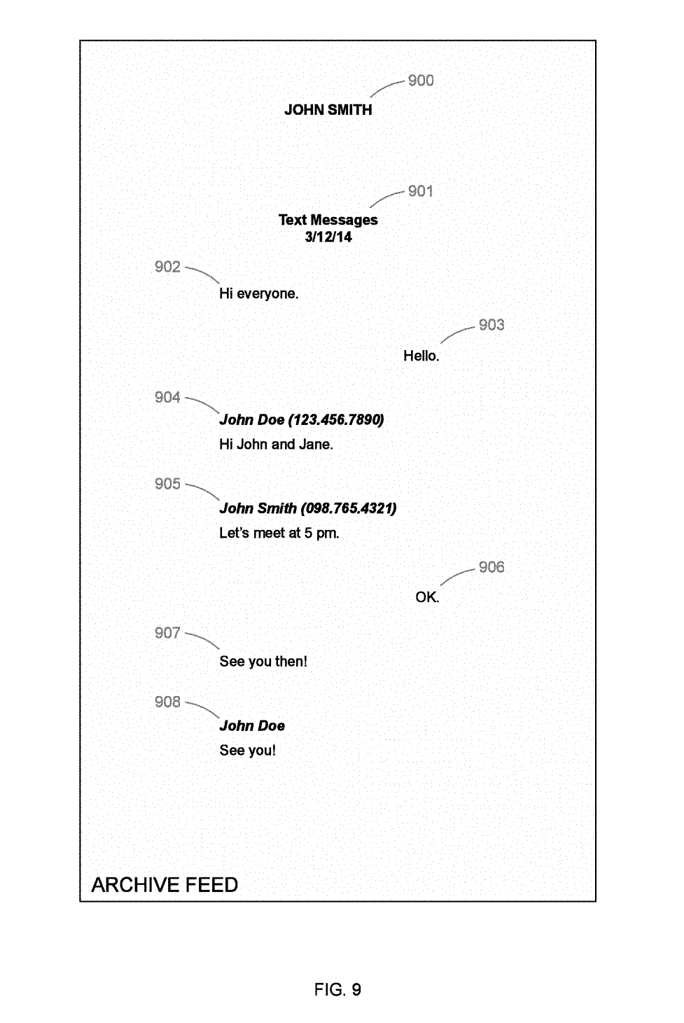

FIG. 9 is a diagram that displays a general strategy for displaying messages in a group conversation. Group conversations are usually found in direct correspondence channels, such as text messages, instant messages, and email. In the interest of maintaining context and clarity for messages exchanged between the user (Jane Doe) and the archive contact (John Smith), the communication data from third-party participants (contacts that are not part the archive, in this case John Doe) in group interactions may be included in each archive.

This diagram shows a text message conversation 901 in a single contact archive for John Smith 900. Messages from the archive contact 902 are not labeled with the sender name by default, as they are displayed distinctly from the user messages 903 (left and right, respectively). Messages from the third-party participants are labeled with the name (and contact information, for example a phone number or email address) 904. The next message from the archive contact should be labeled with the archive contact name (and, optionally, contact information) 905. Labeling the archive contact in this case creates a distinct separation from the third-party participant, which can also be achieved using graphic strategies, such as a dividing line.

Messages exchanged between the user 906 and the archive contact 907 do not need to be labeled with names (see 2.2). Subsequent messages from the third-party participant may include just the name 908, but not the contact information.

There may be group conversations that include both the user and the archive contact(s) in the recipients list, but where neither of the user nor the archive contact(s) participates in the interaction. The archival importance of such conversations may vary, so they can be filtered out either automatically or by offering the user an option to clear these conversations from the archive feed.

2.4 Creating and Sorting Conversations

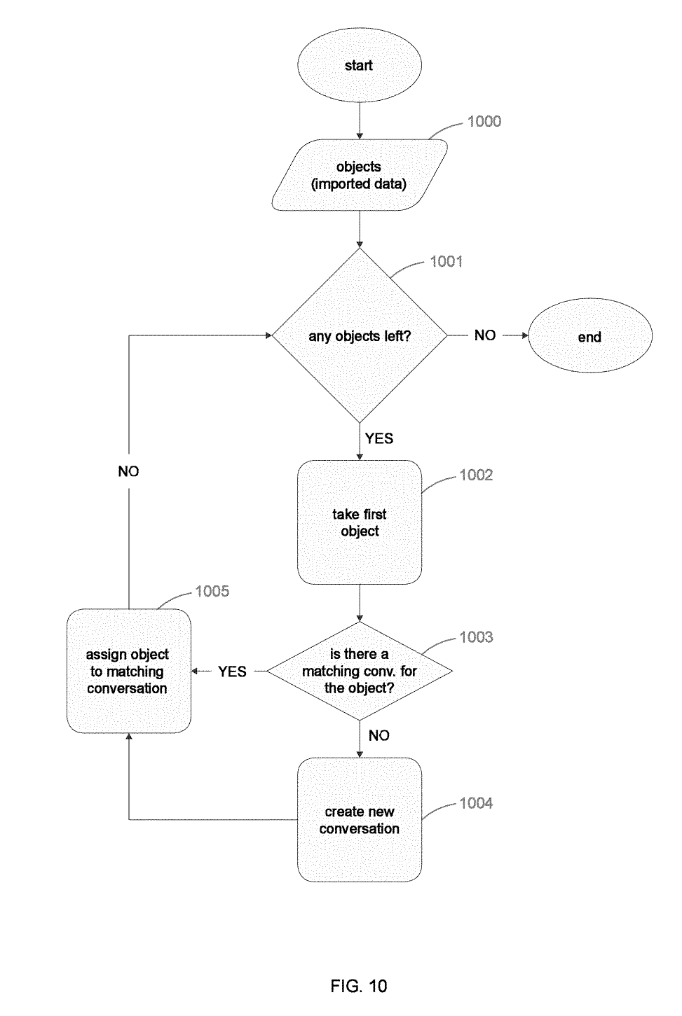

FIG. 10 is a flowchart that describes the conversation creation algorithm, which matches each record to the correct conversation. The System clusters all imported records (“objects”) 1000 into conversation units using one of the methods in FIG. 8. All objects are sorted into conversations according to their channel, period, continuity, and/or other markers. If there are remaining unsorted objects 1001, the System tries to match 1003 the next object 1002 to existing conversations 1005. If there is no matching conversation 1003 for that object 1002, the System creates a new conversation 1004 and assigns the object 1002 to the conversation 1005. This algorithm repeats until there are no more unsorted objects.

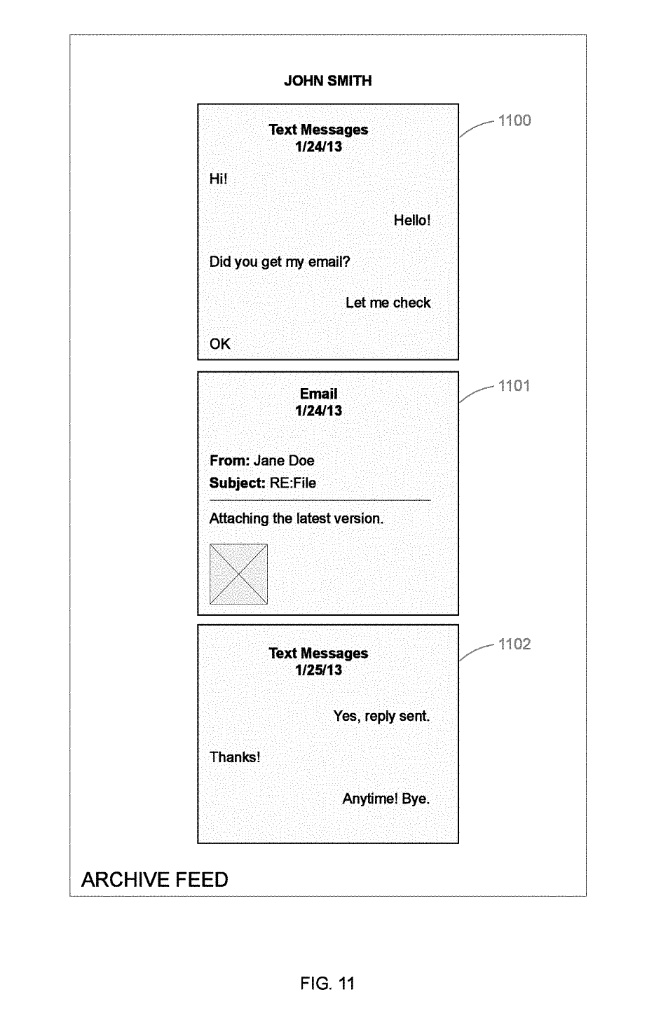

FIG. 11 is a diagram that demonstrates how to best organize conversations in a single timeline (“feed”). In this embodiment, the conversations are clustered in a way that should not disrupt the chronological order of all records.

In some cases, the archive sorts records from multiple channels that could otherwise be clustered into a single conversation. For example, text messages 1100 and 1102 would normally form a single conversation, but an email exchange 1101 occurred in the middle of that interaction. In this case, text messages 1100 and 1102 may be split into two separate conversations and the email 1101 may be placed in between to maintain chronological order. In another example embodiment, the System could disregard the difference in channels.

2.5 Processing for Analytics

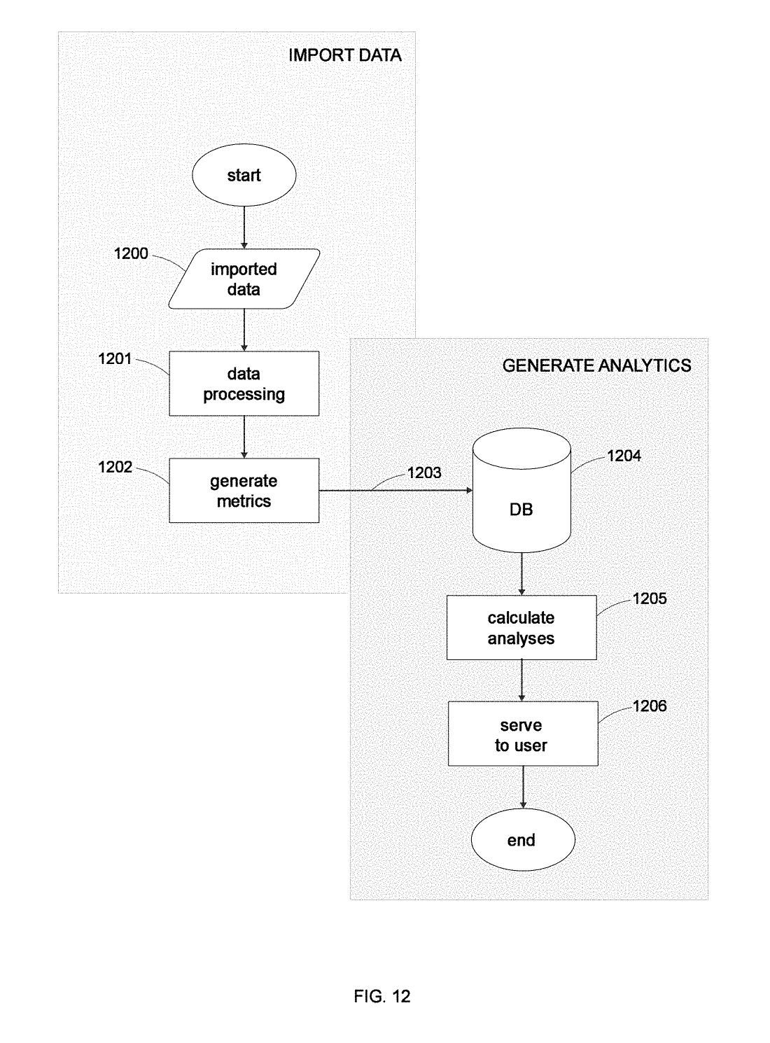

FIG. 12 is a flowchart that describes an example preparation of analytics data. The System takes imported data 1200, and processes them 1201 in order to generate metrics 1202 (primary calculations used in subsequent analyses). Resulting metadata are saved 1203 into database 1204 to prevent the System from repeating the first operations 1201-1202 multiple times.

The metrics metadata are used to calculate a set of “analyses” 1205 (described in Part 4), and the results are prepared and served to the user interface 1206. When the user syncs new data, the system may calculate new metrics 1202, and/or update the existing ones.

Part 3: User Interface

The System may not only offer users a specialized data organization and storage solution, but give each user easy access to their data.

The System can be delivered to the end user in the form of a web application, desktop application (for a desktop or laptop computer), and/or a mobile application (for smartphones, telephones, tablets, and similar devices with suitable application support). The System may utilize only one of these application types to offer its features, or it may create a synchronized network across multiple types. This embodiment may consider a network of all three application types for maximum accessibility, but the description uses the interface of a mobile application to explain the main features.

3.1 User Flow for Creating and Syncing an Archive Profile

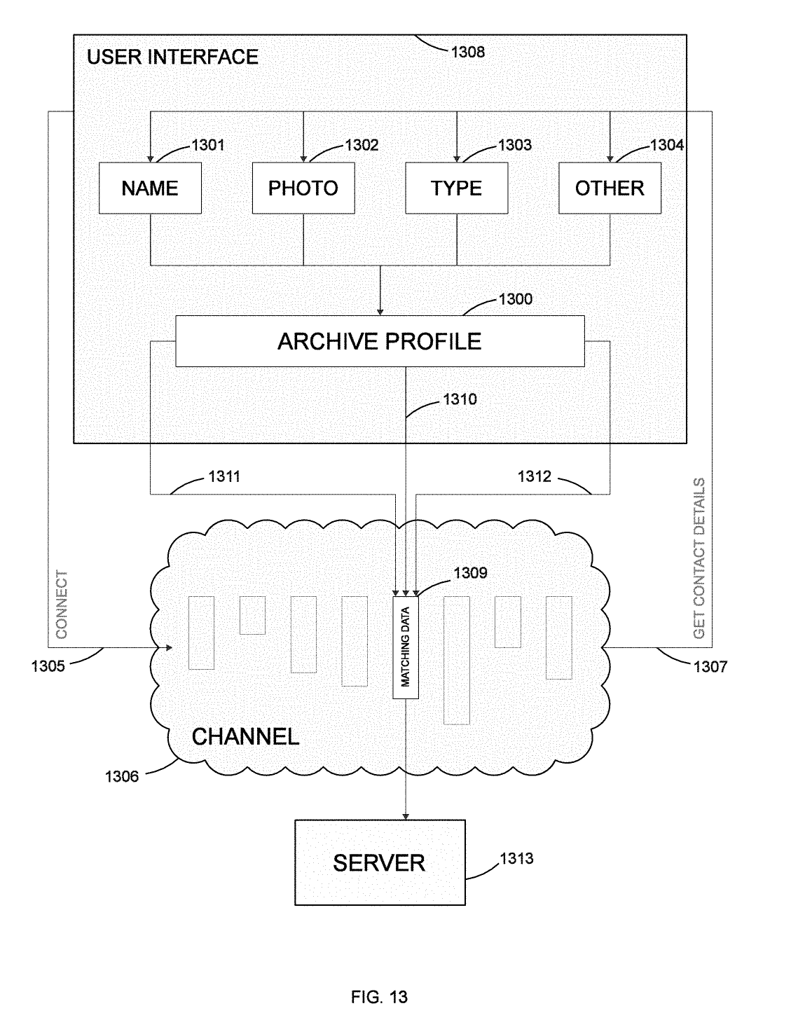

FIG. 13 is a diagram that gives an example of how the user may create and sync an archive. The user begins by creating a profile for the archive contact (“archive profile”) 1300 and adding the archive contact name 1301, photo 1302, relationship type 1303, and any other data 1304. Other embodiments may collect more information for each contact, but the aforementioned 1301-1303 is the minimal set of data for optimal System operations.

The relationship type 1303 allows the System to categorize user's archives for improved display, filtering, analytical, and management features. The System may give the user a predefined list of types (friend, family member, romantic partner, former partner, acquaintance, colleague, etc.) and/or allow the user to enter custom types.

The contact name 1301 may be entered manually by the user, or imported from a contact list, address book, or a social network. To import the name 1301 (or any other information 1302-1304), the user launches the connection 1305 to a channel that keeps this information 1306. The information is downloaded 1307 from the channel 1306 and displayed in the user interface 1308. The user confirms the validity of entered information and completes the archive profile 1300.

Copying, importing, or synchronizing data from a channel requires the user to first select a channel 1306 that holds the data and authorize the System's access 1305 to the channel(s) 1306 (by logging in, or otherwise authorizing the connection to a remote or local source). The user and/or the server may then begin identifying and selecting data for syncing.

Given the complexity of syncing data from multiple channels (as described in 1.2), this embodiment utilizes a data selection strategy that allows for multiple methods of syncing correct data (data that matches the relationship in the archive). This strategy accommodates channels with varying synchronization options using three general approaches:

1. Channels that support contact information 1309 that can be fully matched with the archive profile (contact identifier) 1300. The user verifies an automatic match of the archive contact(s) 1300 to the contact(s) (and/or matching data) 1309 in the channel, then syncs the located data 1310 to the server 1313.

2. Channels that partially support contact information 1309, but are missing some types of information used in the archive profile 1300 (e.g. first name, last name, contact photo). The user manually searches and/or selects the archive contact(s) 1300 from the list of contacts (and/or matching data) 1309 in a channel 1306 (or manually inputs contact details into a search interface), then verifies and syncs located data 1311 to the server 1313.

3. Channels that do not support contact information 1309 and cannot be matched using the archive profile 1300. The user manually selects the data 1309, and then syncs it 1312 to the server 1313.

Depending on the channel, the server 1313 may automatically sync matching data from a channel 1306 given sufficient and/or unique contact identification in the archive profile 1300 that matches the contact information of that channel (for example, an email address or a phone number), which assumes minimal risk of matching error (contacts with identical names, name spelling variation, etc.).

The System may also be set up to sync the entire history from a channel (which may include all data for all available relationships), but the more optimal strategy is to extract only the data that is relevant to the user's needs.

3.2 Archive Feed Design and Functionality

The archive may be a feature of the System, to display a compiled relationship history in an accessible way for the user. An example view of each archive may be the archive feed, which presents all records of the user's interactions with their contact(s). The feed is designed as a chronological transcript that narrates the relationship history using communication data. The best embodiment of the archive feed focuses primarily on clarity and user-friendly design.

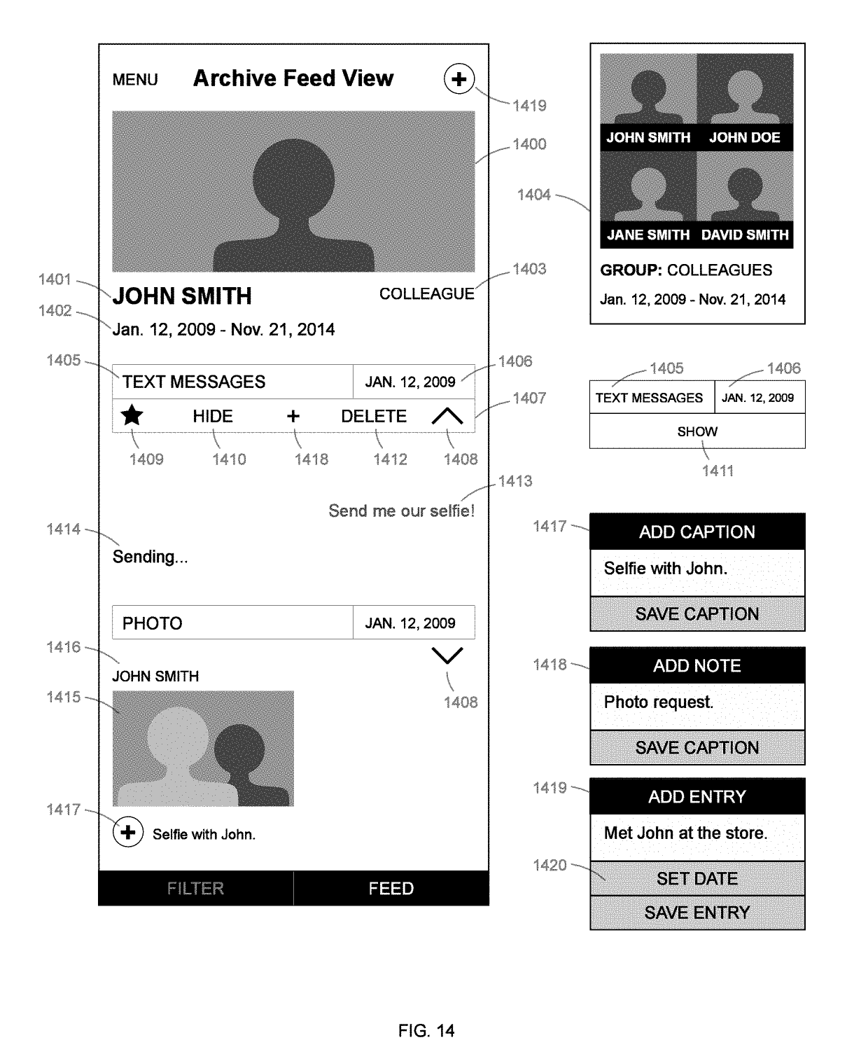

FIG. 14 is a diagram of the archive feed view. The feed contains all synchronized data that has been processed as described in Part 2. Some of the archive profile information can be displayed in the archive, including the profile picture 1400, name 1401, the date range 1402 for the archive contents (dates of the oldest and newest conversations), and relationship type 1403. In the case of multiple contacts in a group archive, there may be a grid of profile pictures or another layout to display each contact and their information 1404.

The archive feed may be presented in a linear timeline of records. In this embodiment, the timeline is clustered by conversations (as described in 2.2), and the description may follow this organizational model. However, each subsequent feature may also be applied to a timeline that uses a different clustering strategy, including an ungrouped timeline of discrete and individually controlled records (individual messages, media, etc.).

This embodiment assumes a clustering method that groups by date (among other parameters), which allows the date to appear only once per conversation. In this embodiment, each conversation begins with the channel of origin 1405 and conversation date(s) 1406.

This embodiment allows user modifications to the data at the conversation level. This description may follow that strategy, but certain embodiments may apply the following features to each record, channel, date, etc. Each conversation may include a menu 1407, which can be collapsed or expanded into view 1408.

This menu allows the user to label a conversation as a “favorite” by tapping an appropriate icon 1309. Each conversation can be hidden (“collapsed”) in the feed by tapping “Hide” 1410 (or an appropriate icon). A collapsed conversation may not display any of the records, but may be represented in the feed with the channel label 1405, date label 1406 and a “Show” button 1411, which would make the conversation visible again.

The user may also delete a single conversation by tapping “Delete” (or an appropriate icon) 1412. The effects of the button can vary depending on the embodiment. Deleting the conversation either erases the corresponding original data from the database or only removes the conversation from future displays in the user interface without erasing the data in the database. Keeping the database copy of original data makes it available for future exporting (as described in FIG. 5).

The conversations may be organized in reverse chronological order in certain embodiments (oldest conversations at the “top” of the feed), and the same order is maintained in the records 1413, 1414, 1415 within conversations. Any kind of order could be used depending on the preferences of the user or system administrator.

Some data formats, including instant and text messages, are conducive to graphic separation (as described in 2.2), so user messages 1413 are displayed on the right side and all other messages 1414 are displayed on the left side. Because other formats can include longer message bodies (such as email), they extend to the full width of the feed and thus require an additional layer of graphic identification. The System can introduce a color code for each relationship type, and all messages from the archive contact(s) would thus be colored distinctly from the user messages. Messages from third-party participants in group conversations may have a separate color from the main participants, as well as the name of the sender. Furthermore, each relationship type may have a designated color, which may then be applied to archive contacts. In certain examples, the colors could be customized or changed by the user.

Media-based conversations display the records 1415 (such as photos) in the feed with the record creator's (or sender's) name 1416. Furthermore, the user may add a caption 1417 to the record 1415 (or edit an imported caption). In addition to captions, the user may add a note 1418 for any conversation, which may subsequently appear with that conversation. The user may also add a text entry 1419 (such as a diary), which may be treated as a separate conversation. A text entry can have a manually set date 1420 to control where it appears in the feed.

3.3 Archive Filters Design and Functionality

Relationship histories can span multiple channels and years, which may result in a substantial amount of data in each archive. The System takes advantage of the unified storage of these records by creating a single control interface for navigating the entire relationship history. The archive filter view (“filter”) allows the user to easily locate records, or customize the way the relationship history is displayed in the feed.

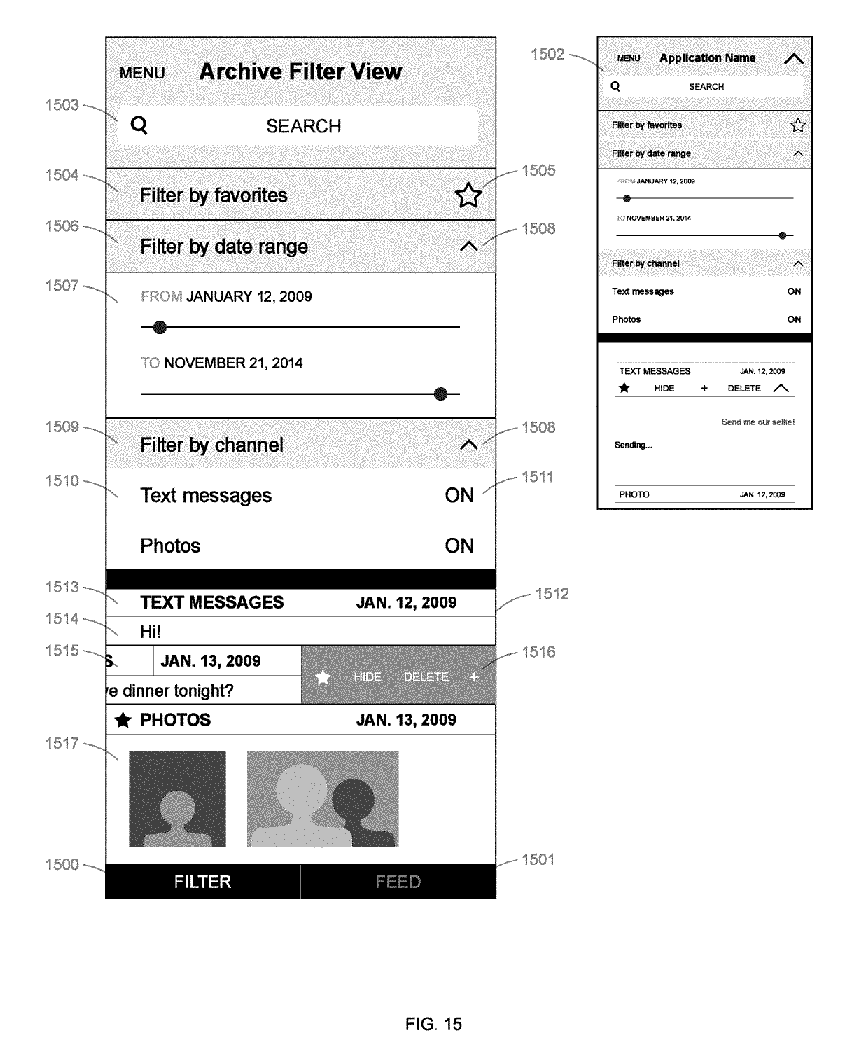

FIG. 15 is a diagram of the archive filter view. In mobile applications, the filter 1500 can exist in parallel to the feed view 1501, so the user can perform a “swipe” gesture or tap to navigate between the two views easily. The filter can also exist within the feed view as a dropdown menu 1502. While the features are identical in both embodiments, this description may focus on the former embodiment. Moreover, the filter can be easily added to the web and desktop applications.

The filter 1500 is primarily an interface that lets the user change the way the feed 1501 is displayed. The user may decide to display only conversations marked as “favorites” 1504 (by tapping an appropriate icon 1505), which may hide other conversations from the feed view until the favorites filter 1504 is disabled. The filter view 1500 can have a compressed preview of all conversations in the archive below the filter menus (“conversations browser”) 1512. The browser 1512 updates along with the feed 1501 whenever the user applies filters 1503, 1504, 1506, 1509.

The user is able to set a custom date range 1506 on all conversations in the archive. This is especially valuable when the user is interested in a particular period. The user sets the start and end dates 1507, which apply to the feed 1501 and the browser 1512 simultaneously. The filter submenu can be expanded and collapsed from view 1508 to maximize the browser 1512.

The user can also filter out certain channels from being displayed in the archive. The filter by channel menu 1509 lists all channels 1510 that have been synced to this archive, and the user may curate the feed display by turning each channel on or off 1511.

The archive is also controlled by a search box 1503, which allows the user to locate conversations (or individual messages, in a different embodiment) that have the matching keyword. Keyword search 1503 is a vital feature in the System, as it allows the user to easily locate records across multiple channels using a single interface. Additional search features, such as keyword suggestions, autocomplete, spell-check, and result counts may also be included to assist the user. As with the other filters, the search results are be immediately reflected in the browser 1512 and the feed 1501.

The user may enable one or more of these filters simultaneously for more advanced settings. These user settings may be retained by the server (see 110 in FIG. 1), or reset each time the user launches an archive.

As a compressed version of the feed 1501, the browser 1512 allows the user to quickly navigate through the conversations and apply the same actions as in the feed 1501. The conversations display the channel and date 1513, plus a preview of the first record (the first lines of a text-based record 1514, or a thumbnail preview of the media records 1517).

The user can “swipe” on a conversation 1515 in the browser 1512 to reveal the conversation menu 1516, with the same features as the conversation menu in the feed view 1501. The menu 1516 allows the user to “favorite”, hide, delete, and add a note to a conversation.

3.4 Archive Freezing

The System is designed to preserve archives of various relationships on the user's behalf. However, the System's objective of making the data easily available to the user may be at odds with that user's short-term needs (for example, if the relationship records are psychologically triggering). In this case, the System offers a feature to temporarily hide (“freeze”) archives. The user can set a timer for a custom or predefined period, which may temporarily remove the archive from the user interface and prevent all user access. Deferring the decision to delete personal records can thus reduce preventable instances of archival data loss.

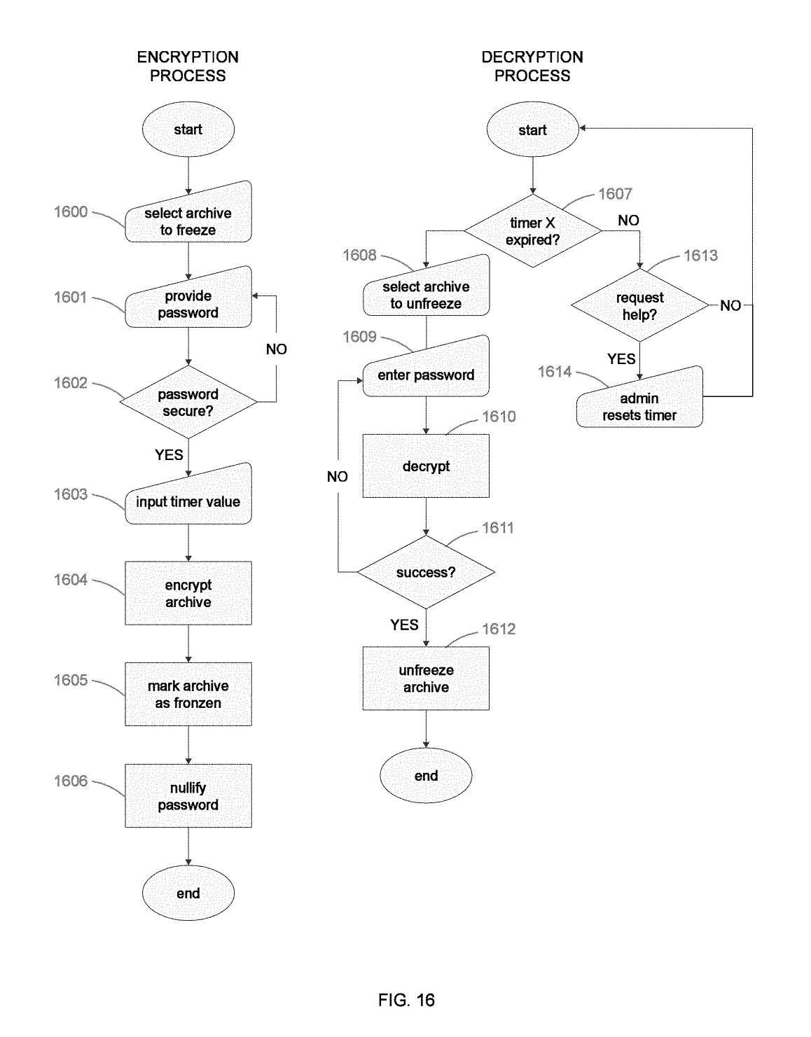

FIG. 16 is a flowchart that describes the freeze archive feature. This flowchart describes one embodiment of the freeze feature, where each archive requires a secured password to unfreeze the archive after the timer ends. The password is not stored in the database, which means the user (or any password-holder) is the only person who can decrypt an archive in such examples. Moreover, if the user (or password-holder) loses the password, the encrypted archive may never be decrypted, even by the System owner/administrator in some examples.

The first process outlines the steps to freeze an archive in such embodiments. The user selects an archive for freezing 1600 and enters a secured password 1601. Upon providing a secure password 1602, the user sets the timer 1603, which immediately encrypts 1604 the archive and marks it as “frozen” 1605 in the System. The secure password provided by the user is nullified 1606 (removed from the System), which secures the frozen archive against System or external access.

The unfreezing process may require the timer to expire 1607. If the timer has expired, the frozen archive may reappear in the user interface, allowing the user to select the archive 1608 to begin the unfreezing process. The user enters 1609 the secure password 1601 and the system attempts to decrypt 1610 the archive. If the password was correct, the decryption may be successful 1611 and the archive may be unfrozen 1612 (restoring user's access to the given archive).

If the timer has not expired, the only way to unfreeze the archive is to request the System administrator or designated person 1613 to reset the timer 1614. A reset timer expires immediately and the archive appears in the user interface, allowing the user to select the archive 1608 and proceed as described above.

Another embodiment of the freeze feature could require no password for freezing and unfreezing an archive. While this embodiment would be user-friendlier, the frozen archive may not be nearly as secure as a password-protected one (as it can be decrypted by anyone with access to the System).

Although a frozen archive won't appear in the user interface, the user may still export the data as part of their complete data package (see 507 in FIG. 5). However, the user may need to provide the input password for each frozen archive to decrypt the data for exporting.

3.5 Archive Library Design and Functionality

In some embodiments, all user archives stored in the System may be accessible via a single interface (“library”). The library is a list and/or gallery view that displays all non-frozen archives to the user. The library view is ideally designed with maximum simplicity to enable easy navigation for the user. The library should also be designed in a way that creates an attractive display of all major contacts in the user's life.

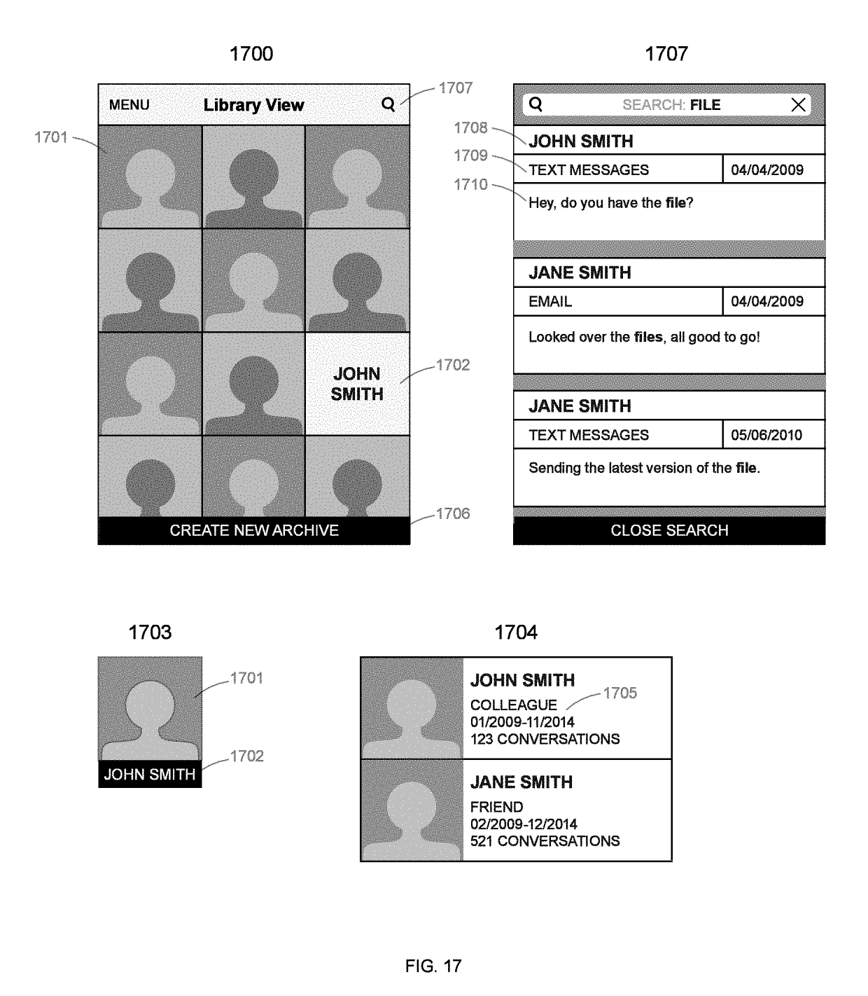

FIG. 17 is a diagram of a library view that displays all archives and includes a library-wide search function. The main library interface 1700 shows archives listed in alphabetical order, although the user may also sort the list differently. Assuming the user knows their contacts by face, the contact name may be dropped from the grid of archives if there is a contact photo (option 1701). If the archive does not have a contact photo, the archive thumbnail may display just the contact name 1702. Alternatively, the library 1700 may be designed 1703 to show both photos 1701 and names 1702 for each archive. Another option 1704 would also include additional archive information 1705, such as relationship type, archive date range, number of conversations, and/or any other relevant information.

The user may initialize the archive creation process from the library (or another navigation interface) by tapping “create new archive” 1706. Additional links and menus can be added to the library view 1700, however they may not obfuscate or detract from the display of archive contacts.

One example feature of the System may be library-wide search 1707. Extending the multi-channel search of each archive, the library-level search further allows the user to locate records from all relationships in the database (with the option to specify which archives to search in). The search box 1707 can be integrated into the main library interface 1700. As in archive search, this search feature allows the user to locate conversations (or individual records, in a different embodiment) across all contacts and channels that match the keyword. The library search interface 1707 may utilize an adapted version of the archive browser interface (FIG. 15) to display condensed results. Each result shows a compressed conversation with the archive contact name 1708, channel and date 1709, as well as the preview of the record 1710 that matches the keyword query.

3.7 Channels Management Interface

The System allows the user to manage data from multiple channels, and relies on connections to these channels to sync data (as described in Part 1). The connection to the channel and the setup process can happen during archive creation, however the System can greatly benefit from a dedicated interface for channel management. This management interface allows the user to connect and disconnect from channels and to manage data imported from those channels.

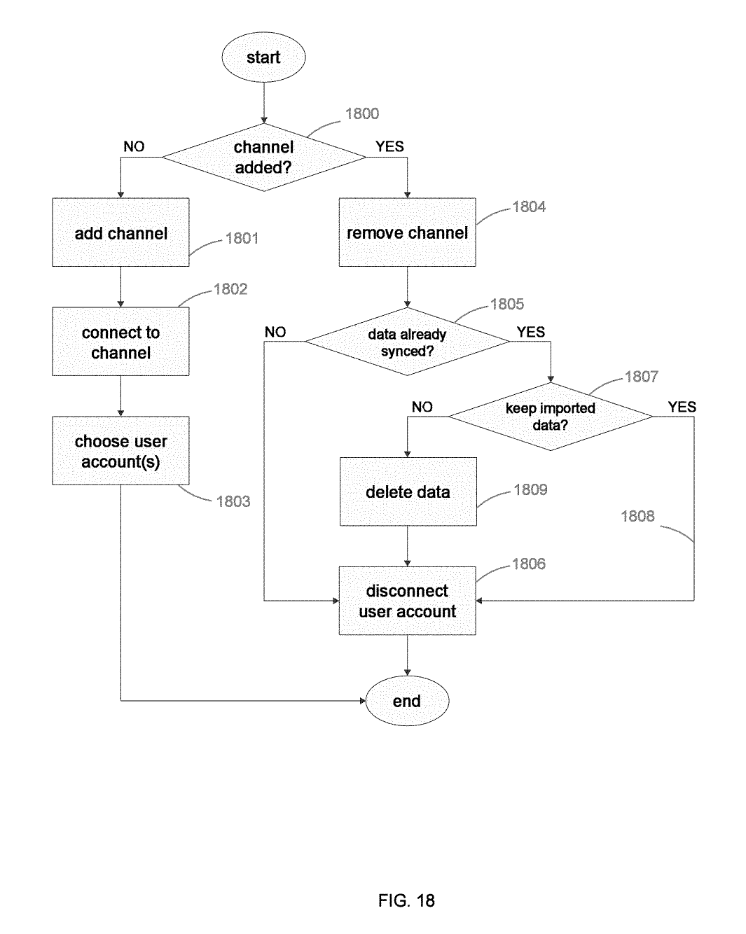

FIG. 18 is a flowchart that describes the channel management process. The user can launch the management interface and review existing channels 1800. If the desired channel is not set up, the user can add that channel 1801 to their account. The server then connects 1802 to the channel (as described in Part 1). Some channels may allow the user to add multiple accounts or identifications within a channel 1803 (e.g. if the user has several email addresses from the same email provider). The channel 1801 saves all settings and is now connected to the user account.

The user can also remove a channel 1804. The process varies depending on previously synced data 1805. If the user has not synced data from the channel, the System disconnects 1806 the user account from that channel. If the user has already synced data from the channel, the System lets the user choose 1807 to keep the previously imported data in the database untouched 1808, but disconnect 1806 the user account from the channel to prevent future syncing. The user may also choose to delete 1809 previously synced data from that channel and then disconnect 1806 their user account from the channel.

3.8 Shared Archives

Some embodiments of the System do not support sharing personal data with external services and other people. However, the System may include an option to create a “shared archive”—an archive managed by two or more users. This is especially practical if two or more users would like to collaborate on their relationship history and to combine everyone's data to make the archive more complete. These users may have equal access to the archive, but have separate analytics and other user-specific user interfaces that the archive inputs into.

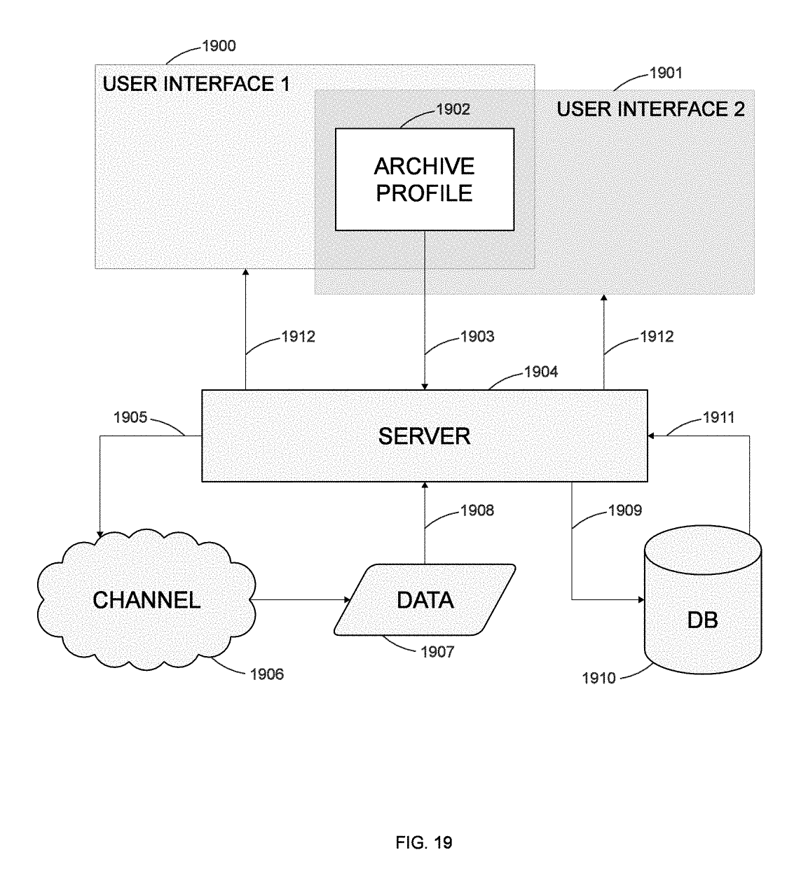

FIG. 19 is a diagram that describes a general setup of a shared archive. Two or more users, using separate user interfaces 1900 and 1901, create a shared archive profile 1902. The archive profile may be set up for both users (the relationship between users 1 and 2), or any third person(s) (the relationship between user 1, user 2, and the third person(s)). The data syncing happens just like in a regular archive, where the profile information is processed 1903 by the System server 1904 and matched with the data in a channel 1906. The imported data 1907 is processed by the server 1904, encrypted, and stored 1909 in the database 1910. When a user wants to access the shared archive, the data is decrypted 1911 from the database and displayed 1912 in that user's personal user interface (1900 or 1901).

Part 4: Communication Data Analytics

The System collects every message, email, photo, and other record for each relationship, which results in the most complete dataset possible of each user's social history. Unlike the raw data scattered across multiple channels, the System database creates a cohesive transcript of each user's social history, which allows for expansive analytical opportunities (“analytics”).

By dealing directly with relationship histories, the System's analytics explore the user's fundamental communication habits and contextualize all relationships on a biographical scale. The analytical insights described below allow users to better understand the nature of their relationships and how they relate to the world. Analytics thus create a biographical tool that summarizes a user's entire social history in one practical interface.

There are numerous possible strategies for achieving the aforementioned goals, however this description may focus on the embodiment that operates at three scales: archive analysis (individual relationships), user analysis (“user profile”—a macro-level view of the user's communication patterns), and library analysis (comparative analysis of all relationships and the macro view of the user's social history).

4.1 Analytics Sequence

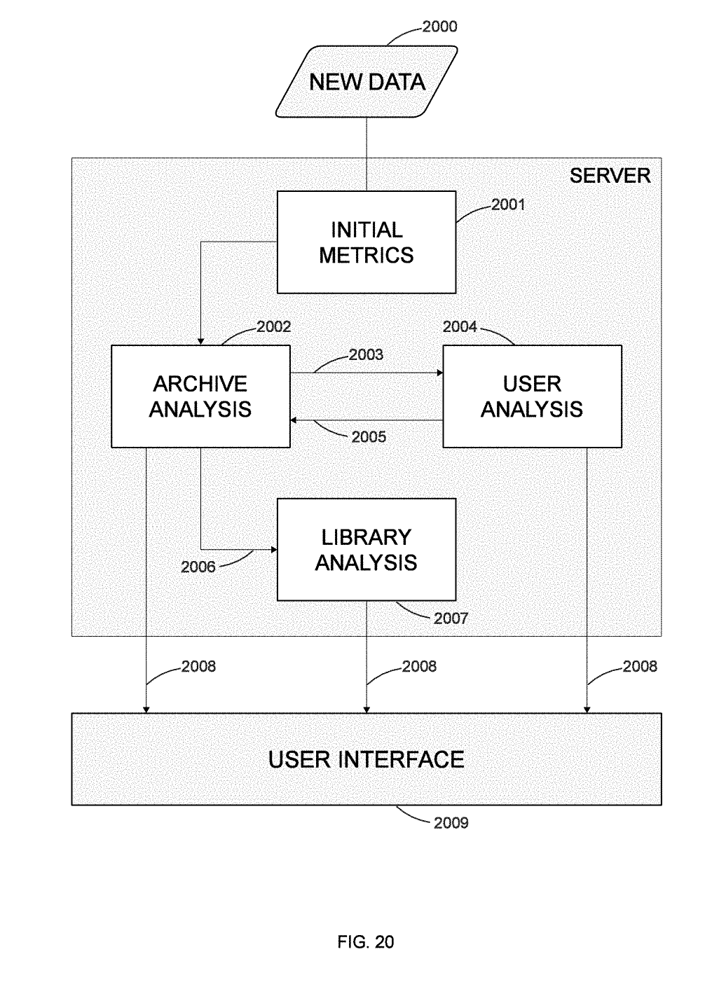

FIG. 20 is a diagram that describes the analytics sequence through all components. The analytics process begins when user syncs new data 2000. The data 2000 is initially processed 2001 (as described in FIG. 12) to form the basis of further analysis. The processing produces initial metrics for each archive separately, to be used in archive analytics 2002. Archive analysis 2002 yields an understanding of the communication patterns between the user and the archive contact(s).

Once the System generates archive analytics 2002 it aggregates the findings and extracts information that relates to the user's personal communication habits 2003, thus creating the user profile 2004. The user profile 2004 analyzes how the user relates to all archive contacts in their library and shows only user-side results. The aggregated user analysis 2004 is fed back 2005 into archive analytics 2002 to generate additional archive analyses, which compare the user's communication patterns in each relationship with the user's macro communication patterns. These additional archive analyses do not feed back into the first (autonomous) archive analyses to avoid cyclical logic.

Finally, all archive analyses 2002 are processed 2006 to create library analysis 2007. Library analytics 2007 give the user a macro overview of all relationships, which includes comparative metrics and other forms of statistical analysis, as well as charts and other visualizations. Library analysis 2007 thus forms the user's analytical map of their entire social history.

The analytics cycle described in this diagram updates every time the user uploads (or removes) new data 2000, which is how the System perfects its analysis of each user's data. Once each analysis component (archive, user, library) is calculated, the results are sent 2008 to the user interface 2009.

4.2 Data Types in Analytics

The System enables users to collect many types of records, which can be generally grouped into three types: direct records of interactions, indirect records of interactions, and manual records. When it comes to analytics, not all data types can be integrated into each analysis. Manual records (such as diary entries, captions, and notes) may not carry the intelligence to analyze interactions between the user and the archive contact. This embodiment may not integrate manual records, although there are possible analyses that can incorporate this data type.

Indirect records of interactions (e.g. photos the user took but didn't share with the contact, event attendance history, the user's gift shopping history, or any form of records that add to the overall reading of the relationship history in the archive feed) may or may not be integrated into the analysis of interactions. The System may choose to integrate certain valuable data, for example shared event history.

Direct records of interactions (including all written messages, social media interactions, call logs, etc.) may be more input examples for analytics and may carry more weight than indirect records. These records transcribe the interactions between the user and the archive contacts, and offer significant analytical intelligence. Although the System may incorporate both manual and indirect records, this embodiment will focus primarily on direct records in the interest of clarity.

4.3 Archive Analysis

Each relationship archive contains an extensive transcript of that relationship. Capitalizing on the transcriptive qualities of digital communications, the System may analyze each record and interaction to produce a macro overview of each relationship. This overview elucidates the relationship history through a series of metrics and visualizations.

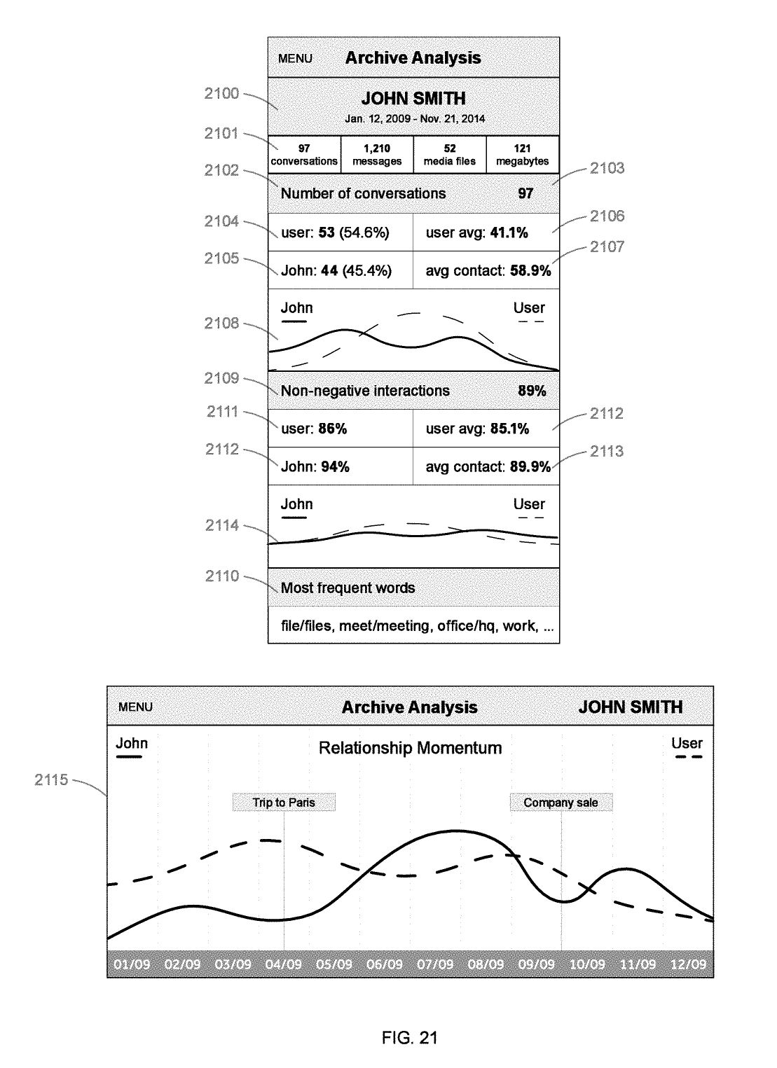

FIG. 21 is a diagram that describes the general elements of an archive analysis, shown in two views. Each archive analysis is created for the archive contact(s) 2100 and features summary statistics 2101, that can include high-level information about the archive, such as the number of conversations, messages, and media files in the archive, as well as storage size.

Interaction analyses 2102 may calculate things like “interaction volume” (number of conversations, messages, etc.) in the relationship, and break the numbers down by overall result 2103, user result 2104, and contact result 2105. As described in FIG. 20, the user profile includes the user's average metrics. These macro-level metrics may be included in the archive analysis to show the user's results across all archives 2106, and the results from all contacts 2107. These averages may be broken up by relationship types to make the comparisons more meaningful.

Finally, each interaction analysis 2102 can include a chart 2108 that shows the change in the analysis results over the duration of the relationship. The charts can be enlarged, for example to full screen, to improve their legibility.

Other interaction analysis can be included in the interface, including “scope” (number of words per message), “responsiveness” (amount of time it takes for the user/contact to reply to messages, and/or the rate of response to messages), average time of day the interactions happened, etc.

The archive analysis can also include linguistic analyses, including algorithms that capture the qualitative metrics, such as the tone of interactions 2109, or more quantitative results, such as the most common words used 2110 or a count of words used. The qualitative metrics 2109 can be presented similarly to interaction analyses 2102, and break down the results by user 2111, contact 2112, macro user average 2112, and macro contacts average 2113. A chart view 2114 is also suggested, to show the changes over time.

All analyses described so far can be further calculated for each channel, for example to see the response rates for text messages, emails, instant messaging, etc. The channel subcategories can be included in the main interface as dropdown elements, to avoid cluttering the high-level results.

Certain analyses may be best presented as only time-based charts, if, for example, they only describe changing patterns over the course of the relationship. Such analyses are best shown either full screen or at an adequately legible scale. For example the chart 2115 shows the analysis of the “relationship momentum”, which captures each person's “initiative” in the relationship's interactions.

The combination of statistical, linguistic, and behavioral analysis in a chart view 2115 can further outline certain relationship markers, such as relationship milestones 2116, conflicts 2117, or any watershed events 2118. These markers provide a useful frame of reference in the mapping of each relationship history.

4.4 User Profile

The System may further process archive analyses to extract user-specific metrics. User metrics may be combined from all relationships to give a macro-level overview of the user's communication habits in a single interface (“user profile”).

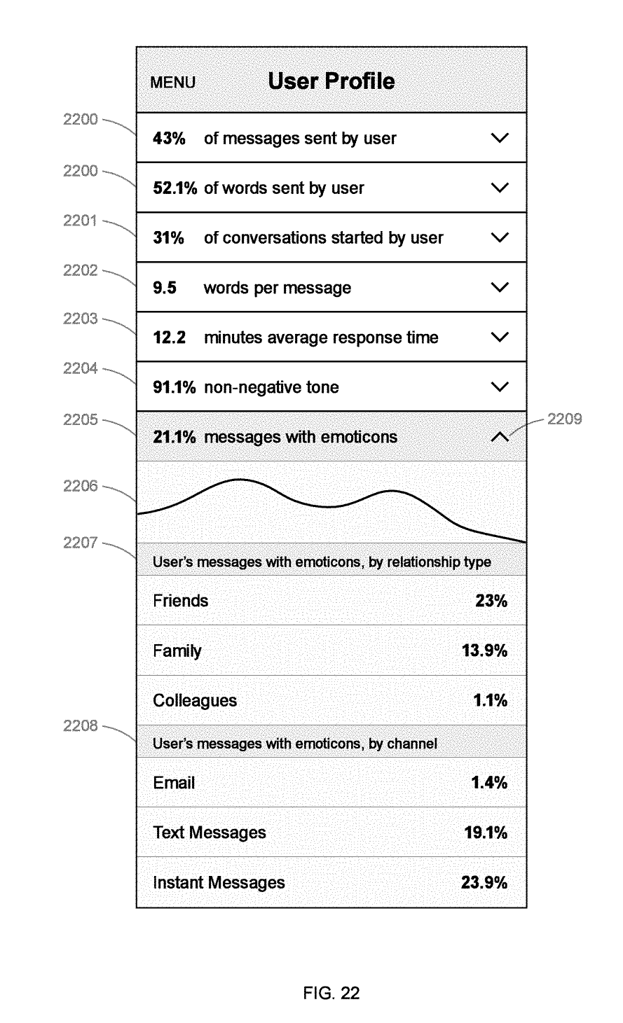

FIG. 22 is a diagram of an example general user profile interface. The aggregated results from all archive analyses are reflected in the user profile, including interaction analyses such as “volume” 2200 (messages and words sent), “initiative” 2201 (percentage of conversations started by user) “scope” 2202 (words per message/messages per conversation), “responsiveness” 2203 (average response time), etc. High-level linguistic analyses are also included, for example the tone of user's communications 2204 and the percentage of user's messages that include certain keywords or emoticons 2205.

It should be noted that tone could be measured using a comparison of words in the communications to lists of words that are categorized as negative, positive, or other type of tone.

Each metric may be accompanied by a chart 2206 that show the changes in the calculation over the course of the user's social history (as stored in the System). The user profile further analyzes each aforementioned metric by relationship type 2207 and channel 2208, which informs the user of any variation across these category types. The chart and subcategory analyses can have a dropdown interface 2209, to avoid cluttering the primary metrics interface.

4.5 Library Analysis

Having calculated archive and user analyses, the System may then process the user's social history at the library level (“library analysis”). The library analysis offers a comparative overview of all relationship metrics across all archives, as well as more complex visualizations of the user's social history.

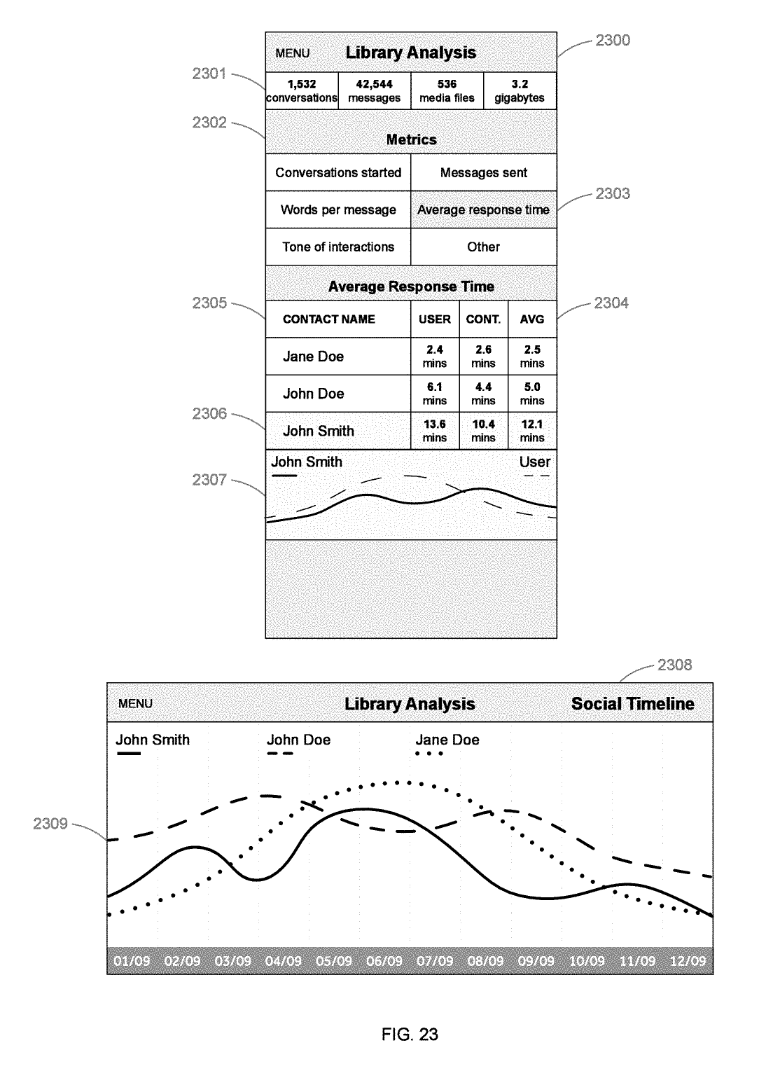

FIG. 23 is a diagram of an example library analysis interface that shows an example of general library analysis 2300 and an example of a macro visualization 2208. The main interface can include summary statistics of the user's account 2301, which can include the number of contacts (by type and in total), conversations, messages, word counts, media files, data usage, etc.

The comparative analyses 2302 give the user an alternative view of some of the metrics in the archive analyses. While the user profile shows the aggregation of archive metrics for the user-specific results, the comparative analyses 2302 combine the analytical results 2304 from different relationships 2005 in a single interface. The user may choose to view an analysis for a particular contact 2306 in a chart 2307.

Combining all relationships in one analytics interface allows the System to display more high-level visualizations that put each relationship in a broader context for the user. A chart that shows the user's social timeline 2308 can map the volume of all interactions over time 2309 (by measuring the most active relationships for each period). Similar high-level visualizations can display an entire social history in a single visualization (or perform and display a calculation across all relationships over time), in effect becoming a biographical map for the user.

These types of visualizations take advantage of all data processing and analytics steps in the System and are the best way to capture macro-level insights about one's social history. Insights from archive, library, and user analyses offer substantial value to the user, as they finally utilize passively stored communication data.

The System analytics may incorporate any number of metadata, statistical, behavioral, linguistic, and other algorithms to extract practical insights for the user based on the user's communication history. This embodiment described several strategies for utilizing communication data for analysis and displaying such results, however the System is not limited to these strategies, designs, or specific analyses.

Part 5: Relationships Manager

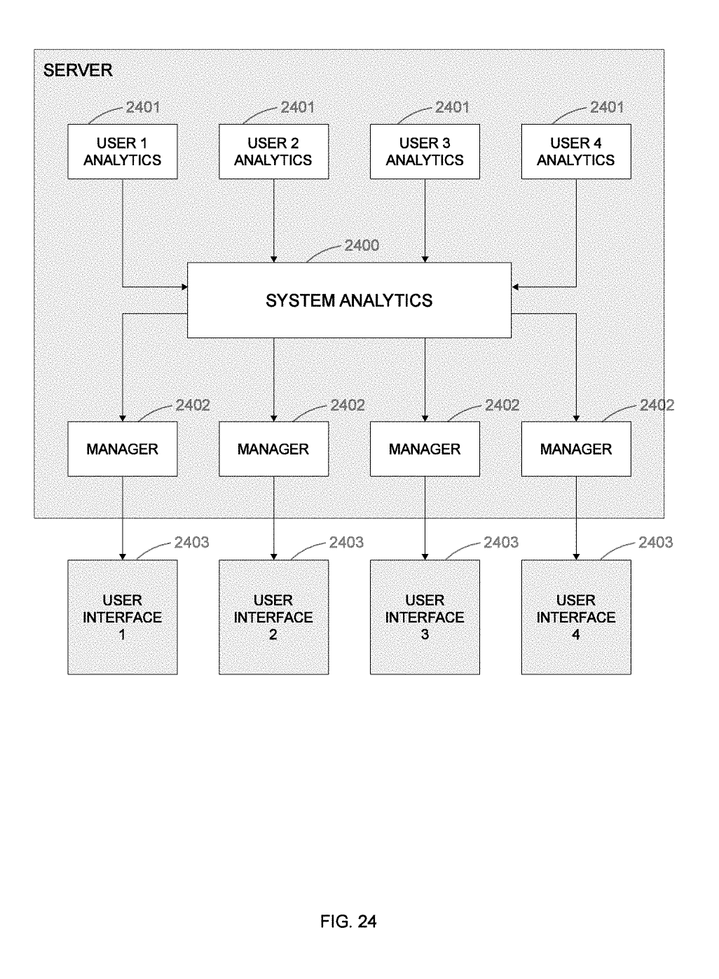

While Analytics looks primarily at past data, the System may offer a tool for managing active relationships and even creating new ones (“relationships manager”, or just “manager”). The manager may be integrated with the user interface and allow the user to better understand and optimize social interactions. The manager may utilize aggregated, anonymized analytics across all users (“system analytics”) to offer not only real-time tracking of each interaction, but also offer predictive assessments based on the user's profile and the entire System database. The manager may offer users advanced insights into their social life, and maximize the latent potential of each user's social history. FIG. 24 is an architecture diagram that describes the general setup of system analytics. System analytics 2400 are a collection of algorithms that may interpret changes in user analytics in real time. System analytics may be up-to-date with each data import and may incorporate many of the calculations and results found in standard user-level analytics (described in Part 4).

The System may extract anonymized data from each user in the system 2401 and processes that data on a system-wide scale 2400. The subsequent analytics output may include a general knowledge base and user-specific results that may be packaged into personalized managers 2402 that are accessible via each user's interface 2403.

5.1 Managing Interactions

The relationships manager may offer a dynamic interface for one's social life by allowing the user to observe the current status of each relationship. Because system analytics mostly input user's own data, much of the status information may sometimes appear as self-evident. However, users may find that certain patterns and changes may be hard to observe from inside the interaction, so the manager as an interface can offer some critical distance.

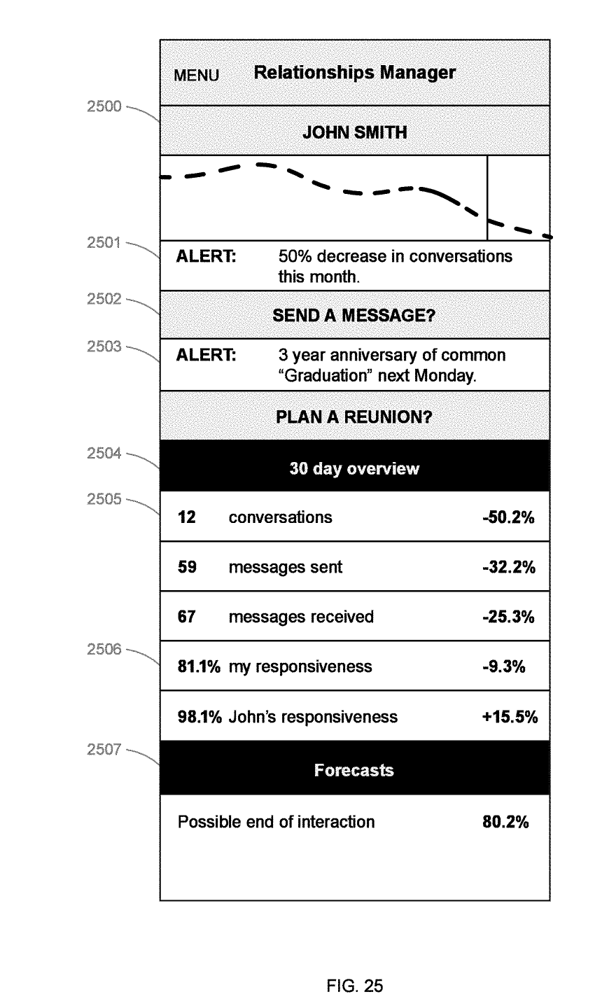

FIG. 25 is a diagram of the relationships manager interface. The manager interface may focus on one archive relationship at a time, so each view could feature information relating to only one contact 2500. The top of the view is reserved for any alerts 2501 that show substantial changes (e.g. a rapid deterioration of interaction frequency or quality). Other alerts may include reminders about upcoming anniversaries 2502 or other milestones.

The manager can offer a suggested action 2503 (e.g. “send a message”), based on the issue at hand, relationship profile and history, user profile, and any System-wide insights. Through system analytics, the System may develop a robust database of communication patterns and measure the causality of certain parameters, which would enable the manager to offer forecasts 2507 (such as warnings of tipping points or suggestions of opportunities).

Alerts can be set up to send a direct notification to the user (e.g. an email, text message, or phone notification). The user can set their settings according to their notification preferences, especially with regards to sensitive relationships that can be “muted” (prohibiting all notifications).

The manager view can also include a communication status report 2504 on some primary metrics, some of which may be an extension of archive analytics. For example, the user could see how the interaction has changed in the past month (or other period) using metrics like messaging volume 2505, changes in the interaction equilibrium 2506 (lopsided communications), increased or decreased communication gaps, changes in the tone of communications, etc.

The relationships manager may ultimately function as a digital secretary, which would offer the user an additional perspective into their day-to-day social lives.

5.2 User-Matching

Taking further advantage of the knowledge base and detailed user profiles, the System can offer features based on inter-user analyses, which compare the profiles of different users and share the results between them. In the most general form, this may operate as a user-matching feature, which analyzes user profiles for compatibility. A compatibility analysis that is based on documented communication histories potentially offers a higher degree of accuracy in capturing the personal profile of a user than most services that rely on self-reported user profiles. Inter-user analysis may thus build a foundation for a new kind of matchmaking technology.

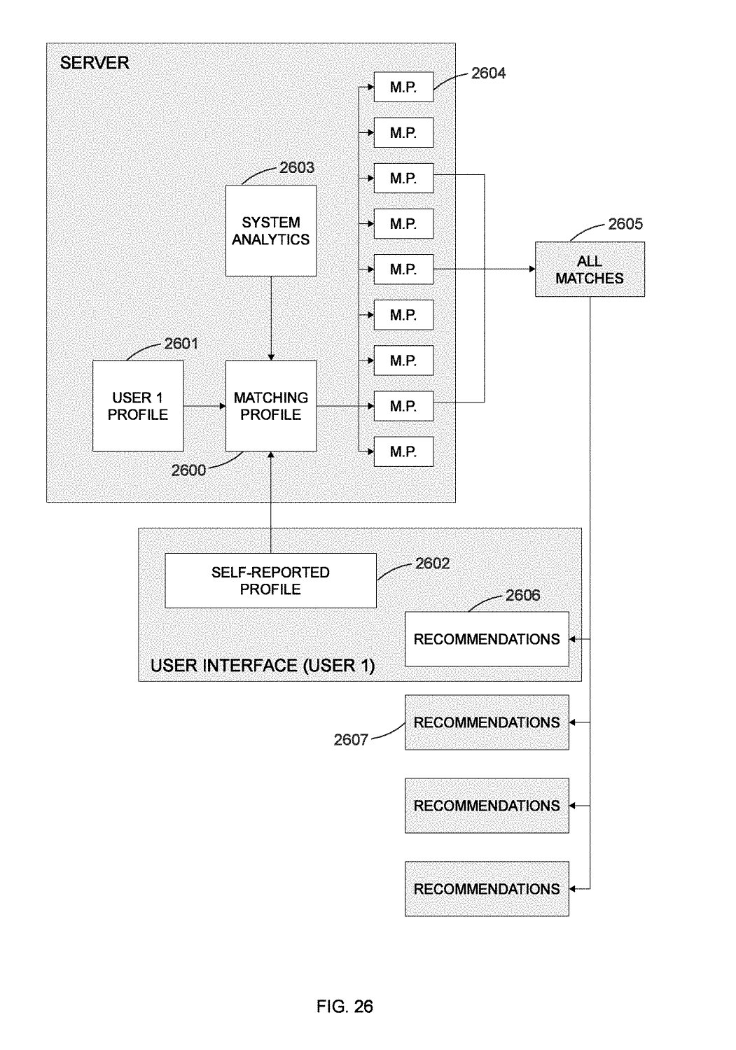

FIG. 26 is a diagram of the general matching process. The core of the compatibility analysis is a user matching profile 2600, which aggregates the analytical outputs 2601 from the user's account, any self-reported data 2602, and system analytics 2603 (the System knowledge base improves the profile calculations by, for example, analyzing the user for standout traits). The user's matching profile 2600 is then analyzed against the matching profile of every other user 2604, using metrics from all analytics calculations, as well as additional algorithms for identifying the best match. A best match could be based on common analysis in the user matching profiles, or analyzed communication data.

Any matches are collected and distributed to all users in the matching pairs in the form of recommendations 2606-2607 that include ways of contacting each other via one of the many channels integrated into the System. Moreover, because the System analyzes each user's profile, it can offer informed recommendations to each user via the manager about how to best connect to each other. This embodiment describes an advanced application of the System capabilities, but in practice all aforementioned features may be fully controlled and opted-into by each user.

5.3 Social Health Tracking

The relationships manager may be further extended to define, estimate, track, and directly or indirectly improve each user's “social health” by comparing communication data analyzed for a user, to a set of pre-defined metrics. Based on the compared data, the system may indicate to a user that certain communication data analysis correlates to a “social health” category. In this way a user may better be able to understand and use the data about their communications, rather than having to contemplate the raw data and interpolate its meaning. The domain of social health may be defined as the study of an individual's social relationships and their effect on one's mental and physical wellness. Just as physical health is typically managed by medical diagnostic practices, so might social health be addressed by the System, thereby creating novel opportunities for improving each person's overall quality of life.

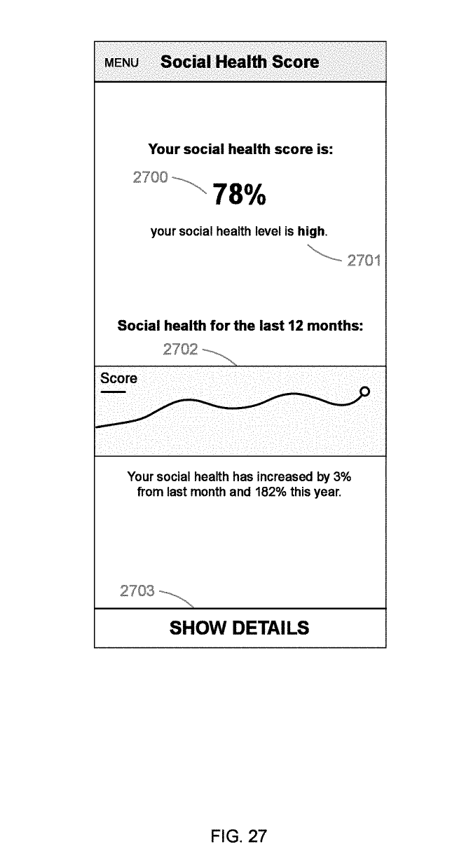

FIG. 27 The System may offer a simple user interface for tracking social health to maximize the benefit to each user and to better encourage the creation of new habits for tracking this type of wellness. In one possible embodiment the System may offer a single (possibly real-time) indicator of overall social health (such as a “social health score”), which may be comprised of any number of factors, parameters, and data analyses that contribute to the estimation of social health.

In this embodiment the output of all relevant social health analyses may be summarized using a social health score 2700 that follows a familiar numeric rating scale, either based on a percentage (0-100%) or an interval scale (0-10, 0-100, etc.). It may be possible to use a graphic rating scale, however a non-numeric embodiment may appear to be less precise and/or practical to the end user. The chosen rating scale may clearly and precisely indicate that user's level of social health, which may be intuitively understood to be “high”, “moderate”, or “low” 2701 (possibly using a more granular classification).

The purpose of the social health score is to efficiently inform the user of the general status of their social health. The design of the score may attempt to cultivate a new habit, whereby the user wishes to regularly check the status of their social health by tracking changes in the health score over time 2702 and/or accessing a more detailed breakdown of social health analyses 2703.

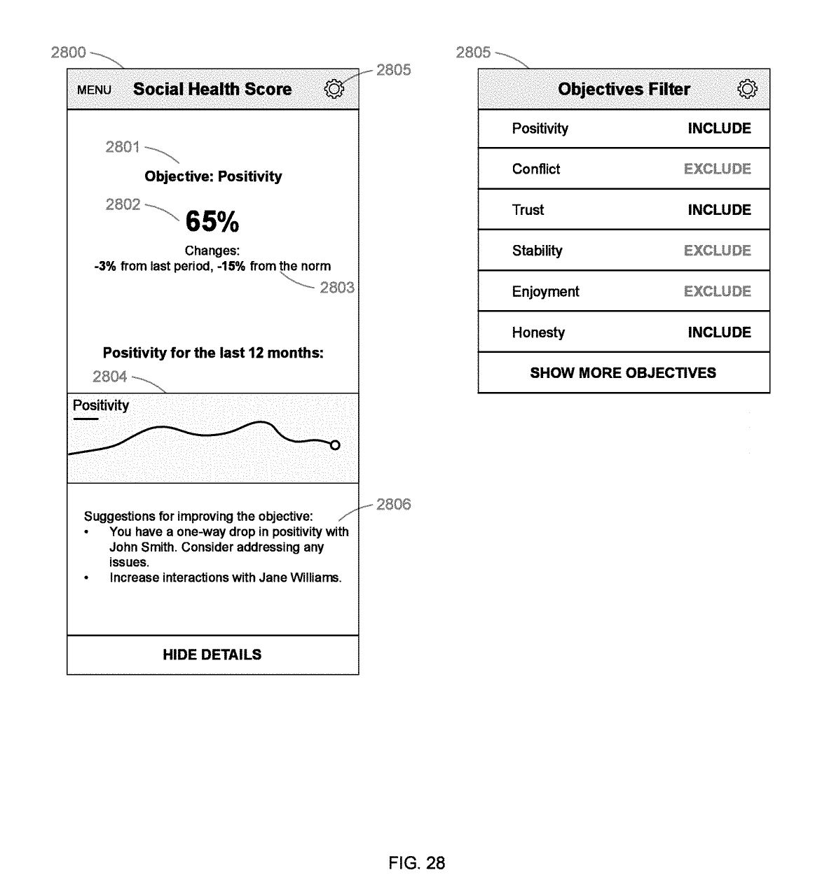

FIG. 28 The detailed view of the relevant social health analyses 2800 may display all available parameters for various personal and/or social objectives 2801, or it may focus only on parameters with significant changes or inconsistent developments. The parameters may be presented as separate scores or metrics 2802 (including any changes from the previous observation period and/or the healthy norm 2803), time series analyses that show historical cycles and fluctuations 2804, or any other format that may best inform the user about a given objective. The System may further give the user some degree of control for displaying their analyses 2805—for example enabling and disabling certain analyses, and/or applying weights, priorities, and other advanced filters and preferences.

Obtaining a rigorous awareness of one's social health may allow a user to define, reinforce, and reach personal, interpersonal, and general “objectives”, both existing and new. Objectives may take the form of various behavioral and personality aspects (such as “intellectual openness”, “emotional sensitivity”, “emotional stability”, “expression of individuality”, “attentiveness”, “likeability”, “honesty”, “positivity”, “optimism”, “assertiveness”, and others) or of aspects that control one's connection to, and involvement with, the outside world (“social engagement”, “trust”, “stable support system”, “diversity of interactions”, “conflict”, “social versus alone time”, “isolation”, and others). The System may also help users to track and screen for various mental health conditions, such as depression and anxiety.

Objectives 2801 may be designed and/or selected by the System, following the recognition, establishment, and/or adaptation of any social health norms and/or reference ranges by the System (based on any internal analytical model and/or other diagnostic frameworks) and/or from outside expertise, to the extent that such norms may assist in the estimation and/or amelioration of a given person's or group's social health.

The System may convey to the user the current and historical (time series) performance of these objectives 2802 (by individual and grouped parameters, while possibly recognizing meaningful combinations, for example “openness” with “stable support system”) and give suggestions for how to reach such objectives 2806 (for example to examine certain relationships or to consider a behavioral change).

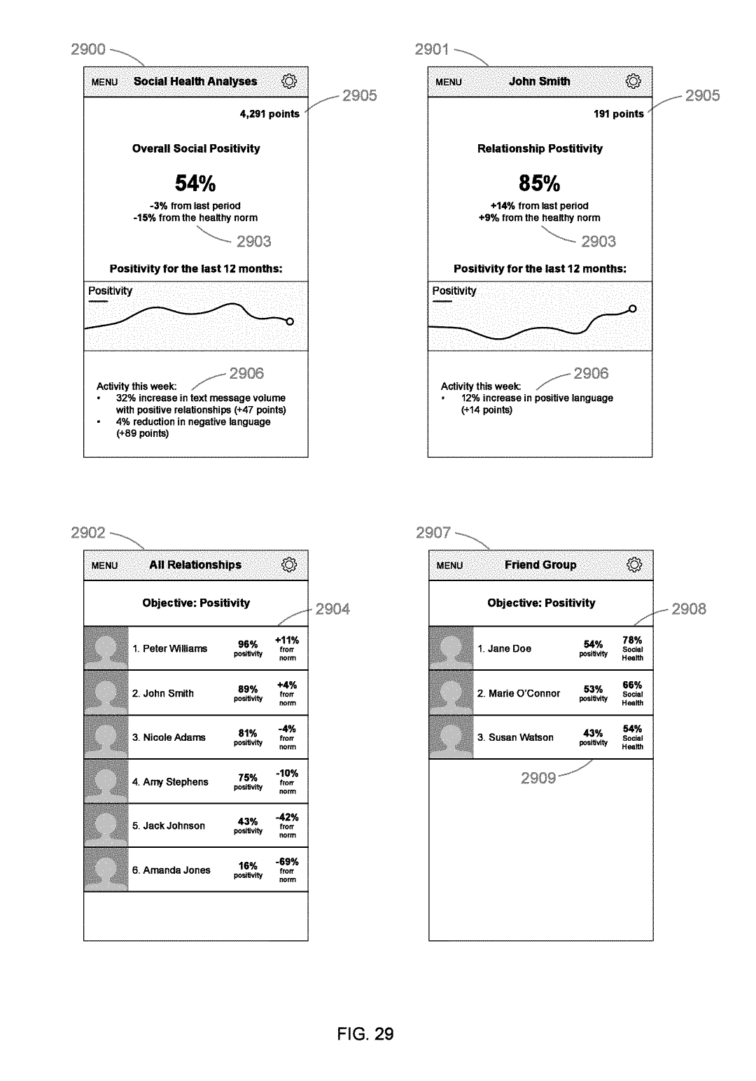

FIG. 29 The success of the System's ability to track social health may rely on the user's active engagement with the service. The System may increase engagement, and the resulting cultivation of healthy habits, by introducing elements of gamification to the service that may prompt a person's natural desire for organization, completion, mastery, learning, competition, and closure.

The social health score (FIG. 27) may become the primary target of self-improvement in this embodiment. However, given its aggregate nature, the social health score may not be immediately responsive to discrete day-to-day actions, which may limit the frequency of feedback to the user.

The System may thus incorporate additional tracking elements into secondary objective analyses (described FIG. 28). Tracking elements may be added to an overall objective analysis 2900, to a relationship objective analysis 2901, and a comparative view of the objective analysis across all relationships of the user 2902.

The tracking elements may contextualize the performance of an objective using established benchmarks and norms 2903, or comparatively—by using internal rankings and/or percentiles based on the person's social history 2904.

The System may further introduce reward elements (points, levels, stars, or other bonus features and classifications) 2905 that directly encourage the user's participation in improving their social health outcomes. For example, the System may track user's social activity for certain events (such as sending a message, making a call, or modifying the nature of certain interactions) and give one or more units of the reward element upon reaching a milestone or carrying out a desirable action 2906.

The reward elements may not contribute to the calculation of the social health score or other objectives, to maintain the accuracy of primary analyses. Rewards and other gamification elements 2906 may only create a secondary feature to enable a user's engagement in the service and interest in improving their social health.

Finally, a social element (such as sharing or displaying one's progress) could be introduced to leverage a sense of support and community that may foster positive behavioral change and help to improve each person's objectives. A user may form or join a group of other users 2907 who may share their social health scores 2908 and/or other objective tracking performance 2909 with each other, similarly to the community element of a physical exercise group.

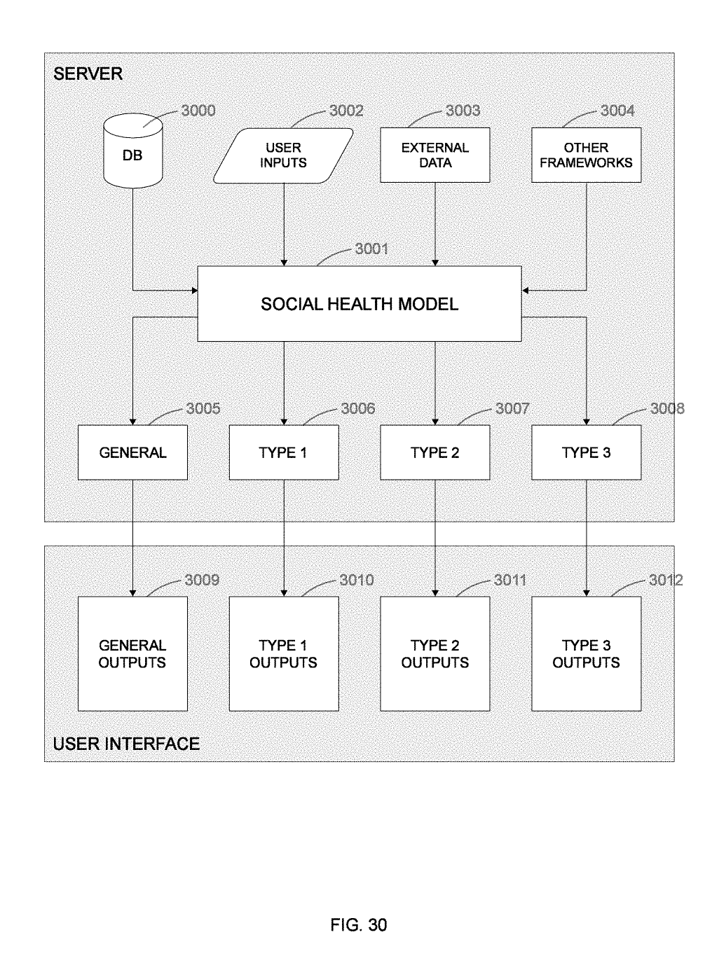

FIG. 30 One component in measuring social health may be the System database 3000, which may collect vast amounts of private social data that may effectively train an analytical model 3001 across a great number of people to validate the known and discover any unknown variables, patterns, and other parameters that control social health. The System may utilize any advanced data science tools and methodologies, particularly neural networks, to process this vast amount of data. The model 3001 may thus pioneer a robust understanding of social (and, by extension, related mental and physical) health etiologies.

The social health model 3001 may be designed to process any number of inputs using any combination of statistical analyses, machine learning, deep learning, and other applications of data science. The System may maximize the model's 3001 accuracy by training it on a substantial variety of inputs, which may divulge previously unknown markers and preconditions for social health. The possible inputs may include the System database 3000, any inputs from the user (such as medical and biographical information, data labels, etc.) 3002, data from external sources (such as social platforms and health tracking tools) 3003, and any other frameworks 3004.

The System may also train separate models for specific objectives. Besides a general model 3005 that may focus on overall social health, the System may include any number of models for each distinct relationship type (such as “family”, “romantic partner”, and “professional”) or another specific objective 3006-3008 for more accurate social health tracking.

The respective outputs 3009-3012 may be served to the user interface in a manner that helps the user understand and manage their social health objectives. A successful embodiment of the System may utilize network effects to maximize the individual value of each person's data by sharing the benefit of the collective social health knowledge with all users.

Part 6. Centralized Social Data Storage Infrastructure

As a result of the proliferation of digital social channels and supplementary technologies, each user may generate increasingly large volumes of personal data, which may be shared with other people, channels, and devices. This process may create a multitude of inefficient copies that carry substantial infrastructural strain, as the vast amount of data requires substantial infrastructural operations to store and maintain each copy of the data. The System may contribute to a long-term reduction in data storage- and sharing-related inefficiencies through simplifying the process of storing, accessing, and sharing of all social data across all channels.

Because the System may be an integrated storage solution for all types of social data it may be offered to third party platforms and various external channels as a centralized solution for the storage, management, analysis, and access of some or all of social data that is created, held, and exchanged by the channels' respective users. The partial or complete centralization of these data operations in a single database (and, possibly, a data processor) may yield significant economic savings and added value for the channel owners, users, and the environment.

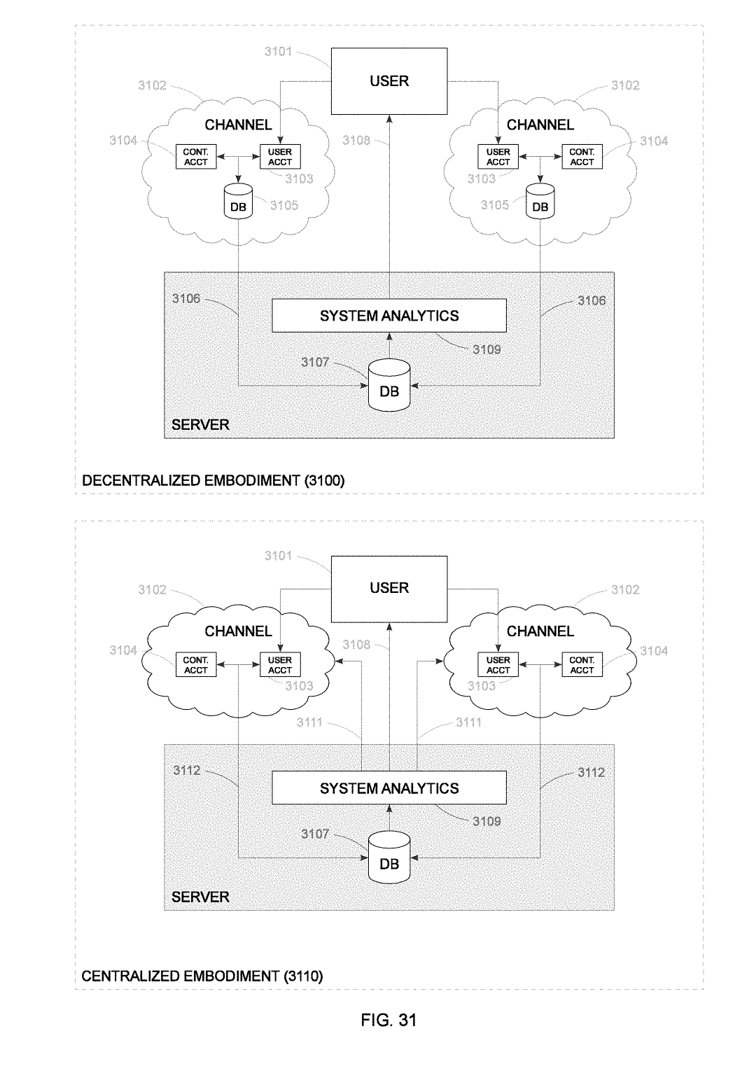

FIG. 31 Under a decentralized embodiment 3100 each user 3101 may engage with a channel 3102 through a user account 3103 to interact with other people (through their accounts on the channel) 3104. Most interactions generate data that is stored in each channel's database 3105. Depending on its size, each channel database 3105 may require a complex, and possibly separate, industrial operation (such as one or more server farms) with high levels of maintenance costs, hardware requirements, human capital, public utility consumption, and environmental pollution.

Moreover, in a decentralized embodiment 3100 users of the System may need to create additional requests (automated or manual) 3106 to move their data from the channel database 3104 to the System database 3107. This suboptimal data copying process may cause users to experience added friction in the data syncing process, undermining the potential benefits from utilizing their data 3108, particularly through System analytics (including all analytical and social health features from this description) 3109.

In some embodiments, a more efficient embodiment may include some degree of centralization 3110. In such embodiments, the System database 3107 and the channels 3102 may be deeply integrated, such that all data generated in each channel 3102 may be stored directly in the System database 3107. The channels 3102 may still access the data as needed for their operations (through any communication protocols), but may no longer be restricted to their own storage operations. Centralizing storage operations around the System database 3107 may offer better economies of scale in operating server infrastructure, and may substantially reduce the amount of economic and environmental waste compared to the decentralized embodiment 3100.

The System database 3107 may be stored in a centralized server infrastructure managed by the System, or through another efficient cloud or networked storage provider. In this embodiment, the System may act as a data processor that enables data centralization, while relying on existing infrastructure.

The channels 3102 may already outsource data storage to an efficient cloud storage provider, in which case the benefits of additional centralization in the System database 3107 may be measured by the reduction of economic and environmental waste from the removal of duplicate user data across various channels and the System. For example, media files may be stored as multiple copies—on the original user device, as shared copies on other people's devices, posts to any social networks, and archived in the System database 3107. The files may be shared via links to the originals, or through any interface that enables centralized access. Any edits to the data (such as filters and captions) may be stored as non-destructive layers or additions to the original files.

In addition to the benefits of a simplified infrastructure and possibly reduced costs of operations, the channels 3102 may be given additional analytics outputs 3111 from the System analytics 3109 to better manage, understand, and serve the users on their channels. This may include insights into users' social behaviors, networks, and other valuable output from the System analytics 3109, subject to prevailing data privacy laws. Furthermore, centralization of data syncing and storage may encourage higher user engagement in the channels 3102, as people may be more inclined to use a channel 3102 that is well-integrated with the System analytics 3109.

The System benefits from the centralized storage by reducing frictions for System users 3101 during data syncing 3112, possibly creating a persistent storage framework for all social data and real-time analytic outputs 3108 across some or all channels 3102. The data is further secured against loss risks compared to the decentralized embodiment 3100 by maintaining a singular database that is designed to process and preserve such data 3107.

Broader benefits of centralization 3110 may include a possible reduction in environmental risks, including lower waste creation, pollution, and need for public utilities such as energy and water. Reducing the data storage needs may also reduce the storage capacity requirements in user devices, which may result in a lower demand for device components and storage devices that contribute to the creation of highly toxic and hard to dispose electronic waste.

Another benefit of a sophisticated data syncing and storage infrastructure may include a more robust System analytical model 3109. Simplifying the flow of information 3112 may create easier System access to more relevant social data, which may boost the accuracy and effectiveness of the System's social health and other analytical analyses. A more complete System database 3107 may generate a general knowledge base for social health that may contribute to broader societal benefits and serve as a foundation for additional innovation.

### CONCLUSION

The System thus reimagines the value of our scattered records of digital communications and considers new applications for their reuse. The systems and methods described here, described in this application map out a new future of social interactions where people choose to preserve and fully utilize their personal communication histories not only for their sentimental value, but also to improve and gain new insights into the nature of all their social interactions.

As disclosed herein, features consistent with the present systems and methods described here, may be implemented via computer-hardware, software and/or firmware. For example, the systems and methods disclosed herein may be embodied in various forms including, for example, a data processor, such as a computer that also includes a database, digital electronic circuitry, firmware, software, computer networks, servers, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the systems and methods described here, or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the systems and methods described here, or it may be more convenient to construct a specialized apparatus or system to perform the required methods and techniques.

Aspects of the method and system described herein, such as the logic, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the systems and methods described here have been specifically described herein, it will be apparent to those skilled in the art to which the systems and methods described here pertains that variations and modifications of the various implementations shown and described herein may be made without departing from the spirit and scope of the systems and methods described here. Accordingly, it is intended that the invention(s) be limited only to the extent required by the applicable rules of law.

The foregoing description, for purpose of explanation, has been described with reference to specific embodiments. However, the illustrative discussions above are not intended to be exhaustive or to limit the invention(s) to the precise forms disclosed. Many modifications and variations are possible in view of the above teachings. The embodiments were chosen and described in order to best explain the principles of the invention(s) and its practical applications, to thereby enable others skilled in the art to best utilize the invention(s) and various embodiments with various modifications as are suited to the particular use contemplated.

What is claimed is:

1. A system for organizing data, comprising:
a computer processor in communication with a data storage server and a network, the computer processor configured to,
communicate with multiple channels over the network,
wherein each of the multiple channels includes stored relationship data and contact information for each of multiple contact identifiers;
receive a designation of each of multiple contact identifiers;
receive an indication of a corresponding channel, from the multiple channels, for each of the designated contact entries;
synchronize relationship data from the corresponding channel for each of the designated contact entries with their corresponding identifier at the channel,
wherein the synchronize includes,
communicate with the corresponding channel through a respective channel interface;
import the stored relationship data and the contact information for each of the multiple identifiers;
store the retrieved relationship data and contact information for each of the multiple identifiers;
relate the stored relationship data and contact information to the respective designated contact identifier; and
update the stored relationship data by retrieving new relationship data and contact information from the respective channel on a regular or irregular basis.

2. The system of claim 1 wherein regular synchronization is periodic scanning for new data.

3. The system of claim 1 wherein an irregular synchronization is triggered by a user-initiated syncing process or new data in the channel.

4. The system of claim 1 wherein a channel is a digital communication channel.

5. The system of claim 1 wherein a channel is a third party social networking web site.

6. The system of claim 1 wherein the system is further configured to cause display of an archive feed related to each of the multiple identifiers.

7. The system of claim 6 wherein the archive feed includes a record of all of the communications with each of the multiple identifiers displayed chronologically.

8. The system of claim 7 wherein the system is further configured to allow a search of the communications data and the multiple identifiers.

9. The system of claim 1 wherein the computer is further configured to store a duplicate copy of the relationship data and contact information from the corresponding channel in the data storage server.

10. A system for archiving digital information, comprising:
a computer including a processor, memory, and data storage server in communication with a network, the computer configured to,
store data regarding contacts and communications for a user in the data storage server;
store an indication of a relationship between the user and each stored contact in the data storage server;
synchronize data over the network by copying user communication data from a channel using a user channel account;
create a user profile for the user and the stored indications of relationships;
analyze the user communication data to determine macro communication patterns;
analyze the user communication data between the user and each stored contact to determine respective micro communication patterns.

11. The system of claim 10 wherein the communication patterns include at least one of, number of words per interaction, amount of time it takes for the user or contact to reply to messages, the rate of response to interactions, and average time of day the interactions happened.

12. The system of claim 10 wherein the communication patterns include sentiment analysis, including analyzing words in the communications to determine at least one of, a tone of communication, keywords used, ideograms, and emoticons used.

13. The system of claim 10 wherein the communication patterns include linguistic analysis, including analyzing words in the communications to determine at least one of, a tone of communication, keywords used, ideograms, and emoticons used.

14. The system of claim 10 wherein the communication matters include message analysis, including counting messages volume, percentage of conversations initiated by a sender, number of words per message, number of messages per conversation, and average response times.

15. The system of claim 10 wherein the computer is further configured to,
compare the communication data for the user to a set of metrics to determine at least one social heath indicator.

16. The system of claim 10 wherein the social health indicator includes at least one of, tone, conflict, trust, stability, enjoyment, depression, anxiety, attachment and reciprocity.

17. A computerized method for organizing data, comprising:
communicating with multiple channels over the network by a computer processor in communication with a data storage server and a network,
wherein each of the multiple channels includes stored relationship data and contact information for each of multiple contact identifiers;
receiving by the computer a designation of each of multiple contact identifiers;
receiving by the computer an indication of a corresponding channel, from the multiple channels, for each of the designated contact entries;
synchronizing by the computer relationship data from the corresponding channel for each of the designated contact entries with their corresponding identifier at the channel,
wherein the synchronize includes,
communicating by the computer with the corresponding channel through a respective channel interface;
retrieving by the computer the stored relationship data and the contact information for each of the multiple identifiers;
storing by the computer the retrieved relationship data and contact information for each of the multiple identifiers;
relating by the computer the stored relationship data and contact information to the respective designated contact identifier; and
updating by the computer the stored relationship data by retrieving new relationship data and contact information from the respective channel on a regular or irregular basis.

18. The method of claim 17, wherein a channel is a third party social networking web site.

19. The method of claim 17, further comprising, by the computer storing a duplicate copy of the relationship data and contact information from the corresponding channel in the data storage server.

20. The method of claim 17, wherein a channel is a digital communication channel
